(19) United States
(12) Patent Application Publication
(10) Pub. No.: US 2024/0298636 A1
(43) Pub. Date: Sep. 12, 2024

(54) ANTIMICROBIAL AGENTS AND FILMS MADE THEREFROM

(71) Applicant: YISSUM RESEARCH DEVELOPMENT COMPANY OF THE HEBREW UNIVERSITY OF JERUSALEM LTD, Jerusalem (IL)

(72) Inventors: Meital RECHES, Kfar Bin Nun (IL); Daniel BOAS, Rishon LeZion (IL)

(21) Appl. No.: 18/557,098

(22) PCT Filed: Apr. 26, 2022
(86) PCT No.: PCT/IL2022/050422

§ 371 Date: Oct. 25, 2023

Related U.S. Application Data
(60) Provisional application No. 63/179,680, filed on Apr. 26, 2021.

(51) Int. Cl. A01N 25/24; A01N 25/10; A01N 59/20; A01P 1/00

(52) U.S. Cl. CPC A01N 25/24; A01N 25/10; A01N 59/20; A01P 1/00

(57) ABSTRACT

The technology subject of the present application concerns antimicrobial and/or antifouling materials and films made therefrom.

### TECHNOLOGICAL FIELD

The invention generally contemplates antimicrobial materials and uses thereof.

### BACKGROUND OF THE INVENTION

The worldwide outbreak of COVID-19, caused by the novel severe acute respiratory syndrome coronavirus 2 (SARS-CoV-2), has drastically affected the health, economy, and quality of life of many countries across the world. COVID 19 is the third emergence in humans of a highly lethal coronavirus since the 2002-2003 SARS pandemic and the ongoing 2012 Middle East respiratory syndrome (MERS) outbreak.

In addition to the coronavirus, the influenza viruses can also cause respiratory infections. These viruses are the cause of four pandemics since the 20th century, including the devastating 1918 Spanish flu and the continuous seasonal outbreaks in the annual period known as the flu season. Although the major transmission pathway of COVID-19 appears to be aerosols, both COVID-19 and influenza, as well as other diseases caused by viruses that infect the respiratory tract, can spread through contaminated surfaces and fomites.

The importance of fighting nosocomial infections, which are mainly caused by bacterial contamination of surfaces, is reinforced as the resistance of these pathogenic bacteria to antibiotics expands. Over 44% of the most frequently reported pathogens across healthcare-associated infections are caused specifically by gram-negative bacteria. Bacterial contamination of surfaces is not a problem present solely in hospitals and medical equipment, rather it can affect any place or object where transmission of these bacteria to humans is possible. These include public transportation, elevator buttons, door handles, keyboards, and touch screens.

The burden of both viral outbreaks and bacterial infections could be mitigated via a single coating with dual functionality, providing both antiviral and antibacterial properties. Different materials were used as a coating that combines antiviral and antibacterial functions, such as silver nanoparticles, TiO₂, small-molecule organics, polymers, and copper-containing surfaces. The antimicrobial mechanism of these materials involves different pathways, such as reactive oxygen species (ROS) production, contact killing, and membrane depolarization by the dissolution of metal ions. Antibiotic resistance usually occurs because the antibiotic targets a single biochemical process. Different copper-based materials display a pleiotropic effect as they involve several antimicrobial pathways, negating the development of antibiotic resistance. The exact antimicrobial mechanism of copper remains uncertain, but several processes have been proposed. Copper ions were shown to bind bacterial cells, causing membrane depolarization that leads to leaks and ruptures. Another process associated with copper ions, which is also apparent in copper nanoparticles (CuNPs), involves the production of ROS that can damage the genetic material and the lipid membranes. Specifically, Cu(II) ions have a lesser effect on viruses while the more potent Cu(I) ions were shown to possess significant antiviral abilities. The antimicrobial abilities of metallic Cu via contact killing were proposed to involve four processes: (i) copper dissolution from the surface which may cause cell damage, (ii) the cellular membrane loses its integrity and its contents, (iii) the copper ions induce ROS formation, and (iv) the genetic material undergoes degradation.

### GENERAL DESCRIPTION

The inventors of the technology disclosed herein have devised a novel class of stable, robust and highly affectatious materials that provide antimicrobial properties, including antiviral, antibacterial and antifungal properties; and films and coatings made therefrom that have effective surface-adherence, transparency and other improved mechanical and biochemical properties.

The novel class of materials disclosed herein encompasses a number of bifunctional compounds having a surface adhering functionality and at least one metal-ion associating functionality. The functionalities may be associated directly or via a linker moiety which itself may contribute not only to the tailored association of the two functionalities but also to an ordered arrangement or assembly of the compounds when used for modifying a surface antiviral, antifungal and/or antibacterial properties. Interestingly, materials of the invention exhibit antifouling properties involving prevention of microorganisms to the material or surface, wherein these properties are achievable even in the absence of a functionality, such as a fluorine atom, aromatic amino acids, and others, known and selected to reduce adhesion of microorganisms to a target material or a surface. Materials of the invention, therefore, provide not only the ability to endow a surface with a continuous and prolonged surface sterility by destroying bacteria, fungi and viruses that come in contact with the surface, but also prevent surface adhesion of microorganisms; hence bestowing surface antiviral, antifungal and antibacterial attributes.

In a first of its aspects, the invention concerns a multifunctional, e.g., at least bifunctional, material, the material comprising at least one surface adhering functionality, at least one organic metal-ion associating functionality, and optionally at least one structurally and/or biologically effective functionality.

The invention further provides an antiviral and/or antibacterial material, the material comprising at least one surface adhering functionality, at least one organic metal-ion associating functionality, and optionally at least one structurally and/or biologically effective functionality.

Also provided is an antimicrobial and/or antifouling material comprising at least one surface adhering functionality, at least one organic metal-ion associating functionality, and optionally at least one structurally and/or biologically effective functionality.

Further provided is a material or an antiviral/antifungal/antibacterial material comprising at least one surface adhering functionality, at least one organic metal-ion associating functionality, and optionally at least one structurally and/or biologically effective functionality, wherein the at least one surface adhering functionality and the at least one organic metal-ion associating functionality are associated directly or indirectly via at least one linker moiety, said linker moiety being optionally the at least one structurally and/or biologically effective functionality.

In some embodiments, materials of the invention exclude such materials having F-based antifouling functionalities such as fluorinated alkyls, fluorinated aryls, and F atoms.

Materials (or interchangeably-compounds) of the invention are antiviral and/or antifungal and/or antibacterial materials that may be presented as compounds of the general formulae:

A-M, or (1)

A-L-M, or (2)

A-M-T, or (3)

T-A-M, or (4)

A-T-M, or (5)

T-A-L-M, or (6)

A-L-T-M, or (7)

A-T-L-M, or (8)

A-L-M-T; (9)

wherein A is at least one surface adhering functionality, M is at least organic metal-ion associating functionality, L is at least one linker moiety, T is at least one structurally and/or biologically effective functionality and “-” is at least one bond, as disclosed herein. Each of the aforementioned formulae constitutes a separate and independent embodiment of the invention.

As depicted in structure (1) above, the at least one surface adhering functionality (A) is directly associated via a chemical bond, which may be a covalent or an ionic bond, to the at least one organic metal-ion associating functionality (M). In cases where at least one structurally and/or biologically effective functionality (T) is present, it may be associated to the surface adhering functionality (A), as depicted in structure (4) above, or it may be associated to the metal associating functionality (M), as depicted in structure (3) above. Alternatively, the structurally and/or biologically effective functionality (T) may bridge the surface adhering functionality (A) and the metal-associating functionality (M), as shown in structure (5).

Independent of whether or not functionality (T) is present, a linker moiety (L) may be introduced to associate any two functionalities together. As depicted in structures (2) and (6)-(9), the linker moiety (L) may be used to associate functionalities (A) and (M), or (A) and (T), or (M) and (T).

In some embodiments, the linker moiety (L) may be functionality (T).

Typically, all functionalities may be associated to each other via covalent bonds (i.e., “-” designates a covalent bond), and where a linker moiety (L) is present, association of each of the functionalities to the linker moiety may be via covalent bonds as well. Thus, in some embodiments, all “-” may designate covalent bonds.

The “at least one surface adhering functionality” is at least one atom or a group of atoms capable of secure association with a surface region. The association or adherence refers to any physical or chemical interaction to be formed between the functionality and the surface region. The association may involve Van-der-Walls, coordinative, covalent, ionic, electrostatic, dipole-dipole, or hydrogen bonding or association or interaction.

The functionality permitting surface adherence is typically a hydroxyaryl moiety, wherein an aryl group has two or three or four or five hydroxy groups. These are referred to as di-, tri-, tetra-, or penta-hydroxyaryls, respectively.

In some embodiments, the aryl is a phenyl ring, or a substituted phenyl ring, or a fused phenyl ring, or a biphenyl ring system.

In some embodiments, the hydroxyaryl is catechol-based functionality, i.e., a phenyl having two ortho hydroxyl groups.

In some embodiments, the surface adhering functionality is a catechol-based functionality.

In some embodiments, the catechol is 3,4-dihydroxy-L-phenylalanin (DOPA).

In some embodiments, the surface adhering functionality is DOPA.

In some embodiments, the surface adhering functionality or the hydroxyaryl is 3,4-dihydroxy-L-phenylalanin (DOPA). In some embodiments, the hydroxyaryl is a hydroxylated DOPA, namely a DOPA having one or more hydroxy groups (in addition to the two hydroxy groups already present in the DOPA molecule). These hydroxylated DOPA compounds may be hydroxy-DOPA (having overall three OH groups), dihydroxy-DOPA (having overall 4 OH groups) or trihydroxy-DOPA (having overall 5 OH groups).

In some embodiments, the hydroxyaryl is ortho, meta or para dihydroxyaryl; 1,2,3-, 1,2,4-, 1,3,4-, 1,3,5-trihydroxyaryl; 1,2,3,4-, 1,2,3,5-, 1,3,4,5-, 1,2,4,5-tetrahydroxyaryl; or 1,2,3,4,5-pentahydroxyaryl, wherein the numbering is used to designate the position of each of the hydroxy groups relative to another hydroxy group on the aryl ring, e.g., phenyl ring.

In some embodiments, the surface-adhering functionality is DOPA or a moiety comprising DOPA. In some embodiments, the moiety comprising DOPA is an organic moiety selected from amino acids and aliphatic functionalities. In some embodiments, the organic moiety is an amino acid. In another embodiment, the organic moiety is a peptide.

In some embodiments, the surface-adhering functionality is DOPA being linked, associated or bonded to an atom of the linker moiety L, as further defined herein.

The “at least one organic metal-ion associating functionality” is at least one organic functionality that comprises an atom or a group of atoms (which may be different from carbon) capable of reversibly associating (and dissociating) to a metal ion baring any valency or charge. The functionality is selected amongst such which are capable of holding the metal ion and release it under predetermined conditions. The functionality may be selected to hold the metal ion under certain conditions and release the metal ion once such conditions are altered. Such conditions may be hydration and dehydration, acidic or basic or neutral pH, ionic strength, presence or absence of better chelating agents, oxidative or reductive conditions, thermal conditions, irradiation under certain wavelengths, etc.

Suitable metal-ion associating functionalities may include an atom selected from sulfur, nitrogen, oxygen, phosphorus, and others, each being associated or bonded to an organic linker or group. In some embodiments, the atom is provided as a group of same or different atoms.

In some embodiments, the functionality is at least one organic group comprising one or more sulfur atoms, and/or nitrogen atoms, and/or oxygen atoms, and/or phosphorus atoms.

In some embodiments, the organic group is an amino acid or a peptide.

In some embodiments, the amino acid or peptide is selected amongst such having at least one pendent nitrogen group. Non-limiting examples include histidine, arginine, proline and lysine.

In some embodiments, the amino acid is histidine.

In some embodiments, the histidine is provided as a histidine-comprising peptide, wherein 3 or more histidine units are provided in chain (e.g., at least -His-His-His . . . ). In some embodiments, the number of histidine units is between 3 and 10, 3 and 9, 3 and 8, 3 and 7, 3 and 6, 3 and 5 or 3, 4, 5, 6, 7, 8, 9 or 10 histidine units. In some embodiments, the number of histidine units is 6.

In other words, the metal-ion associating peptide functionality may be of the structure -(His)n, wherein “-” designates a bond to a linker moiety or to another functionality in a compound of the invention, “His” is histidine and n designates a number between 3 and 10, as defined. For example, where n is 3, the metal-ion associating peptide functionality is -HisHisHis. Where n is 6, the metal-ion associating peptide functionality is -HisHisHisHisHisHis.

In some embodiments, the metal-ion associating functionality is derived from ethylenediaminetetraacetic acid (EDTA), nitrilotriacetic acid (NTA), pyridine and amino terminal Cu(II)- and Ni(II)-binding (ATCUN) motifs.

In another aspect, the invention provides a material comprising at least one surface adhering functionality, at least one organic metal-ion associating functionality comprising between 3 and 10 histidine amino acid units or comprising a peptide functionality consisting between 3 and 10 histidine units, and optionally at least one structurally and/or biologically effective functionality.

The invention further provides a material having antimicrobial properties, namely antiviral, antifungal and/or antibacterial properties, the material comprising at least one adhering functionality, at least one organic metal-ion associating functionality consisting between 3 and 10 histidine amino acid units, and optionally at least one structurally and/or biologically effective functionality.

In some embodiments, the metal-ion associating functionality is selected based on the metal ion it is designated to associate with and release.

In some embodiments, the number of histidine units is 3, or 4, or 5, or 6, or 7.

Materials of the invention may be provided free of a metal ion or may be provided with at least one metal ion associated with the metal-ion associating functionality. Thus, in some embodiments, materials of the invention, as disclosed herein, comprise at least one surface adhering functionality, at least one organic metal-ion associating functionality comprising between 3 and 10 histidine amino acid units or comprising a peptide functionality consisting between 3 and 10 histidine units, and optionally at least one structurally and/or biologically effective functionality, wherein the at least one organic metal-associating functionality is provided with a metal ion.

The metal ion may be a metal ion of any valency or charge, e.g., +1, +2, +3, or higher, that can inhibit microbial growth, e.g., by killing the microorganisms, by decreasing their growth rate, or by causing a stasis in the growth rate. In some configurations, the metal ion exerts its effect, e.g., antibacterial effect, when in association with the metal-ion associating functionality. In some configurations, the metal ion is released from the material into the environment or is deposited on the surface to which the material adheres.

The metal ion may be selected from Cu metal ions, Ni metal ions, Au metal ions, Zn metal ions, Ag metal ions, Mg metal ions, Ti metal ions, Co metal ions, Zr metal ions, Sn metal ions, Pb metal ions, Mo metal ions and others or combinations of several different metal ions.

In some embodiments, the metal ion is selected from Cu⁺¹, Cu⁺², Ni⁺², Ag⁺¹, Ag⁺², Zn⁺², Au⁺², Mg⁺², Ti⁺², Ti⁺⁴, Co⁺², Co⁺³, Zr⁺¹, Zr⁺², Sn⁺², Sn⁺⁴, Pb⁺², Pb⁺⁴, Mo⁺⁴, Mo⁺⁶ and others.

The association between the metal-ion associating functionality, e.g., a plurality of histidine amino acid units, and the metal ion may be ionic or coordinative. In some embodiments, the metal-ion associating functionality forms a coordinative association (or a complex) with the metal ion. Such coordinative associations are known in the art.

Thus, in some embodiments, materials of the invention, as disclosed herein, comprise
at least one surface adhering functionality,
at least one organic metal-ion associating functionality comprising between 3 and 10 histidine amino acid units or comprising a peptide functionality consisting between 3 and 10 histidine units, wherein the metal-ion associating functionality coordinatively associates a metal ion optionally selected from Cu metal ions, Ni metal ions, Au metal ions, Zn metal ions, Ag metal ions, Mg metal ions, Ti metal ions, Co metal ions, Zr metal ions, Sn metal ions, Pb metal ions, Mo metal ions and combinations thereof, and
optionally at least one structurally and/or biologically effective functionality, being in some embodiments, an aromatic amino acid or an amino acid containing at least one pendent nitrogen-containing or nitrogen-based group.

In some embodiments, materials of the invention, as disclosed herein, comprise
at least one surface adhering functionality,
at least one organic copper-ion associating functionality comprising between 3 and 10 histidine amino acid units or comprising a peptide functionality consisting between 3 and 10 histidine units, and
optionally at least one structurally and/or biologically effective functionality.

The present invention further concerns a material as disclosed herein, as a complex with at least one metal ion, as described herein.

A ligand-metal ion complex is further provided, the complex comprising
at least one surface adhering functionality,
a ligand-metal-ion complex of at least one organic metal-ion associating functionality comprising between 3 and 10 histidine amino acid units or comprising a peptide functionality consisting between 3 and 10 histidine units (being the ligand) and a metal ion, and
optionally at least one structurally and/or biologically effective functionality.

The “at least one structurally and/or biologically effective functionality” is any atom or a group of atoms that is selected based its structure and/or chemical/physical properties to contribute a certain structural stability, 3D-arrangement, or special orientation to the compound and/or to contribute one or more biological attributes such as antifouling attributes.

In some embodiments, the at least one structurally and/or biologically effective functionality is an aromatic amino acid. In some embodiments, the aromatic amino acid is phenyl alanine or a derivative thereof, each as disclosed herein.

In some embodiments, the at least one structurally and/or biologically effective functionality is an amino acid having at least one pendent nitrogen group. Non-limiting examples include histidine, arginine, proline and lysine. In some embodiments, the amino acid is lysine. In some embodiments, the amino acid, e.g., lysine, representing a functionality T is provided bonded to the at least one surface adhering functionality, e.g., DOPA. Thus, in some embodiments, a material of the invention is of a structure DOPA-Lys-M, wherein M is as defined herein.

The “linker moiety” constituting an indirect means to associate any two of the functionalities disclosed herein, may or may not be present in compounds of the invention. Linker moiety (L) may be an atom or a group of atoms which enable association of one functionality to another, or any number of functionalities to another functionality. In other words, the linker moiety (L) may be configured for association of two functionalities, e.g., an alkylene having two bonds available for covalent association (such as —(CH₂)n-), may be configured for association of three functionalities, e.g., an atom having three bonds available for covalent association (such as a N atom) or any other moiety capable of associating one or a plurality of functionalities. The linker moiety may thus be an organic moiety, an inorganic moiety or a heteroatom (such as S, N, O, P).

In some embodiments, the linker moiety is an organic moiety.

In some embodiments, the organic moiety is selected from a substituted or unsubstituted carbon chain, an amino acid or a peptide of 1 to 50 amino acids, or 1 to 20 amino acids, or 2 to 20, or 2 to 10 amino acids or 2, 3, 4, 5, 6, 7, 8, 9, 10 amino acids.

In some embodiments, the linker moiety comprises two or more amino acids.

In some embodiments, where the linker is a peptide of two or more amino acids, each of the amino acids may be the same or different.

In some embodiments, the linker moiety comprises an aromatic amino acid.

In some embodiments, the linker moiety comprises at least two amino acids, one or more of the amino acids is an aromatic amino acid.

The term “amino acid” as used throughout the present application, unless otherwise specified refers to any natural or unnatural amino acid, an amino acid analog, α- or β-forms, or may be in either L- or D configurations. Amino acid analogs which may be used in a compound of the invention may be chemically modified at either or both C-terminal and/or N-terminal; or chemically modified at a side-chain functional group (e.g., positioned at the α-position or any other pendant group).

The amino acid may be selected amongst alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine valine, pyrrolysine and selenocysteine; and amino acid analogs such as homo-amino acids, N-alkyl amino acids, dehydroamino acids, aromatic amino acids and α,α-disubstituted amino acids, e.g., cystine, 5-hydroxylysine, 4-hydroxyproline, a-aminoadipic acid, a-amino-n-butyric acid, 3,4-dihydroxyphenylalanine, homoserine, α-methylserine, ornithine, pipecolic acid, ortho, meta or para-aminobenzoic acid, citrulline, canavanine, norleucine, d-glutamic acid, aminobutyric acid, L-fluorenylalanine, L-3-benzothienylalanine and thyroxine.

In some embodiments, the amino acids are selected amongst aromatic amino acids. Non-limiting examples of aromatic amino acids include tryptophan, tyrosine, naphthylalanine, and phenylalanine. In some embodiments, the amino acids are phenylalanine or derivatives thereof.

In some embodiments, the phenylalanine derivatives is 4-methoxy-phenylalanine, 4-carbamimidoyl-1-phenylalanine, 4-chloro-phenylalanine, 3-cyano-phenylalanine, 4-bromo-phenylalanine, 4-cyano-phenylalanine, 4-hydroxymethyl-phenylalanine, 4-methyl-phenylalanine, 1-naphthyl-alanine, 3-(9-anthryl)-alanine, 3-methyl-phenylalanine, m-amidinophenyl-3-alanine, phenylserine, benzylcysteine, 4,4-biphenylalanine, 2-cyano-phenylalanine, 2,4-dichloro-phenylalanine, 3,4-dichloro-phenylalanine, 2-chloro-penylalanine, 3,4-dihydroxy-phenylalanine, 3,5-dibromotyrosine, 3,3-diphenylalanine, 3-ethyl-phenylalanine, 3,4-difluoro-phenylalanine, 3-chloro-phenylalanine, 3-chloro-phenylalanine, 2-fluoro-phenylalanine, 3-fluoro-phenylalanine, 4-amino-L-phenylalanine, homophenylalanine, 3-(8-hydroxyquinolin-3-yl)-1-alanine, 3-iodo-tyrosine, kynurenine, 3,4-dimethyl-phenylalanine, 2-methyl-phenylalanine, m-tyrosine, 2-naphthyl-alanine, 5-hydroxy-1-naphthalene, 6-hydroxy-2-naphthalene, meta-nitro-tyrosine, (beta)-beta-hydroxy-1-tyrosine, (beta)-3-chloro-beta-hydroxy-1-tyrosine, o-tyrosine, 4-benzoyl-phenylalanine, 3-(2-pyridyl)-alanine, 3-(3-pyridyl)-alanine, 3-(4-pyridyl)-alanine, 3-(2-quinolyl)-alanine, 3-(3-quinolyl)-alanine, 3-(4-quinolyl)-alanine, 3-(5-quinolyl)-alanine, 3-(6-quinolyl)-alanine, 3-(2-quinoxalyl)-alanine, styrylalanine, pentafluoro-phenylalanine, 4-fluoro-phenylalanine, phenylalanine, 4-iodo-phenylalanine, 4-nitro-phenylalanine, phosphotyrosine, 4-tert-butyl-phenylalanine, 2-(trifluoromethyl)-phenylalanine, 3-(trifluoromethyl)-phenylalanine, 4-(trifluoromethyl)-phenylalanine, 3-amino-L-tyrosine, 3,5-diiodotyrosine, 3-amino-6-hydroxy-tyrosine, tyrosine, 3,5-difluoro-phenylalanine and/or 3-fluorotyrosine The end C- or N-termini of the peptide may be modified to affect or modulate (increase or decrease or generally change) one or more property of the peptide, e.g., a structural change, hydrophobicity/hydrophilicity, charge, solubility, surface adhesion, toxicity to organisms, biocompetability, resistance to degradation in general and enzymatic degradation in particular and others. The C- or N-termini of the peptide may be chemically modified by forming an ester, an amide, or any other functional group at the desired position; such that the peptides may have an amine at one end thereof (the N-terminal) and a carboxyl group (the C-terminal) at the other end or may have other groups at either of the termini.

In some embodiments, the linker moiety or the amino acid part of any of the aforementioned peptides is phenelyalanine. In some embodiments, the number of phenylalanine groups is two or more.

In some embodiments, the linker moiety L is an amino acid or a peptide comprising 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 or 31 or 32 or 33 or 34 or 35 or 36 or 37 or 38 or 39 or 40 amino acids.

In some embodiments, the linker moiety L has a backbone selected from a substituted or unsubstituted carbon chain which may be saturated or unsaturated, having only single bonds, hydrocarbons comprising one or more double bonds, or one or more triple bonds, or comprising any one or more functional groups which may be pendent to the backbone moiety or as an interrupting group (being part of the backbone).

In some embodiments, the backbone comprises one or more inner-chain aryl groups.

In some embodiments, the linker moiety is an organic backbone moiety selected from substituted or unsubstituted oligomers (having between 2 and 11 repeating units) or polymer (having at least 12 repeating units).

In some embodiments, the backbone may comprise between 1 to 40 carbon atoms or hydrocarbon groups or any heteroatom which is positioned along the backbone (in the main chain). In some embodiments, the backbone comprises between 1 to 20 carbon atoms, or between 1 to 12 carbon atoms, or between 1 to 8 carbon atoms. In some embodiments, the backbone comprises 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 or 31 or 32 or 33 or 34 or 35 or 36 or 37 or 38 or 39 or 40 carbon atoms.

In some embodiments, the linker moiety is constructed of a predetermined number of repeating units which may or may not be randomly structured along the backbone. The linker moiety may be substituted by one or more functional groups such as substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted cycloalkynyl, substituted or unsubstituted aryl, substituted or unsubstituted heteroaryl, substituted or unsubstituted heterocyclyl, substituted or unsubstituted —NR₁R₂, substituted or unsubstituted —OR₃, substituted or unsubstituted —SR₄, substituted or unsubstituted —S(O)R₅, substituted or unsubstituted alkylene-COOH, and substituted or unsubstituted ester. Each of the abovementioned groups is as defined hereinebelow.

The variable group denoted by “R” (including any one of R₁, R₂, R₃, R₄, R₅) independently refers to one or more group selected from hydrogen, alkyl, alkenyl, alkynyl, cycloalkyl, cycloalkenyl, cycloalkynyl, aryl, heteroaryl, heterocyclyl, halogen, alkylene-COOH, ester, —OH, —SH, and —NH₂, as defined herein or any combination thereof. Each of the abovementioned groups, as indicated, may be substituted or unsubstituted. The substitution may also be by one or more R, selected from hydrogen, alkyl, alkenyl, alkynyl, cycloalkyl, cycloalkenyl, cycloalkynyl, aryl, heteroaryl, heterocyclyl, halogen, alkylene-COOH, ester, —OH, —SH, and —NH₂. In some embodiments, the number of R groups may be 0 or 1 or 2 or 3 or 4 or 5 or 6 or 7 or 8 or 9 or 10 or 11 or 12 or 13 or 14 or 15 or 16 or 17 or 18 or 19 or 20.

The invention further provides any one compound of the following:
DOPA-(His)y, wherein His is histidine and y is an integer between 3 and 10,
(DOPA)x-(His)y, wherein x is an integer between 1 and 3, His is histidine and y is an integer between 3 and 10,
(DOPA)x-(AAA)m-(His)y, wherein x is an integer between 1 and 3, AAA is an aromatic amino acid, m is an integer between 1 and 3, His is histidine and y is an integer between 3 and 10,
DOPA-(Phe)p-(His)y, wherein Phe is phenylalanine, p is an integer between 1 and 3, His is histidine and y is an integer between 3 and 10,
DOPA-HisHisHis, wherein His is histidine,
DOPA-HisHisHisHis, wherein His is histidine,
DOPA-HisHisHisHisHis, wherein His is histidine,
DOPA-HisHisHisHisHisHis, wherein His is histidine,
DOPA-HisHisHis-Metal ion, wherein His is histidine, and wherein the Metal ion is as selected herein, e.g., Cu,
DOPA-HisHisHisHis-Metal ion, wherein His is histidine, and wherein the Metal ion is as selected herein, e.g., Cu,
DOPA-HisHisHisHisHis-Metal ion, wherein His is histidine, and wherein the Metal ion is as selected herein, e.g., Cu,
DOPA-HisHisHisHisHisHis-Metal ion, wherein His is histidine, and wherein the Metal ion is as selected herein, e.g., Cu,
DOPA-L-HisHisHis, wherein His is histidine, and wherein L is a linker moiety, as defined herein,
DOPA-L-HisHisHisHis, wherein His is histidine, and wherein L is a linker moiety, as defined herein
DOPA-L-HisHisHisHisHis, wherein His is histidine, and wherein L is a linker moiety, as defined herein
DOPA-L-HisHisHisHisHisHis, wherein His is histidine, and wherein L is a linker moiety, as defined herein
DOPA-L-HisHisHis-Metal ion, wherein His is histidine, L is a linker moiety, as defined herein, and wherein the Metal ion is as selected herein, e.g., Cu,
DOPA-L-HisHisHisHis-Metal ion, wherein His is histidine, L is a linker moiety, as defined herein, and wherein the Metal ion is as selected herein, e.g., Cu,
DOPA-L-HisHisHisHisHis-Metal ion, wherein His is histidine, L is a linker moiety, as defined herein, and wherein the Metal ion is as selected herein, e.g., Cu,
DOPA-L-HisHisHisHisHisHis-Metal ion, wherein His is histidine, L is a linker moiety, as defined herein, and wherein the Metal ion is as selected herein, e.g., Cu,
DOPA-(Phe)p-HisHisHis, wherein His is histidine, Phe is phenylalanine, and wherein p is an integer between 1 and 3,
DOPA-(Phe)p-HisHisHisHis, wherein His is histidine, Phe is phenylalanine, and wherein p is an integer between 1 and 3,
DOPA-(Phe)p-HisHisHisHisHis, wherein His is histidine, Phe is phenylalanine, and wherein p is an integer between 1 and 3,
DOPA-(Phe)p-HisHisHisHisHisHis, wherein His is histidine, Phe is phenylalanine, and wherein p is an integer between 1 and 3,
DOPA-(Phe)p-HisHisHis-Metal ion, wherein His is histidine, Phe is phenylalanine, p is an integer between 1 and 3, and wherein the Metal ion is as selected herein, e.g., Cu,
DOPA-(Phe)p-HisHisHisHis-Metal ion, wherein His is histidine, Phe is phenylalanine, p is an integer between 1 and 3, and wherein the Metal ion is as selected herein, e.g., Cu,
DOPA-(Phe)p-HisHisHisHisHis-Metal ion, wherein His is histidine, Phe is phenylalanine, p is an integer between 1 and 3, and wherein the Metal ion is as selected herein, e.g., Cu,
DOPA-(Phe)p-HisHisHisHisHisHis-Metal ion, wherein His is histidine, Phe is phenylalanine, p is an integer between 1 and 3, and wherein the Metal ion is as selected herein, e.g., Cu,
(Phe)p-DOPA-HisHisHis, wherein His is histidine, Phe is phenylalanine, and wherein p is an integer between 1 and 3,
(Phe)p-DOPA-HisHisHisHis, wherein His is histidine, Phe is phenylalanine, and wherein p is an integer between 1 and 3,
(Phe)p-DOPA-HisHisHisHisHis, wherein His is histidine, Phe is phenylalanine, and wherein p is an integer between 1 and 3,
(Phe)p-DOPA-HisHisHisHisHisHis, wherein His is histidine, Phe is phenylalanine, and wherein p is an integer between 1 and 3,
(Phe)p-DOPA-HisHisHis-Metal ion, wherein His is histidine, Phe is phenylalanine, p is an integer between 1 and 3, and wherein the Metal ion is as selected herein, e.g., Cu,
(Phe)p-DOPA-HisHisHisHis-Metal ion, wherein His is histidine, Phe is phenylalanine, p is an integer between 1 and 3, and wherein the Metal ion is as selected herein, e.g., Cu,
(Phe)p-DOPA-HisHisHisHisHis-Metal ion, wherein His is histidine, Phe is phenylalanine, p is an integer between 1 and 3, and wherein the Metal ion is as selected herein, e.g., Cu,
(Phe)p-DOPA-HisHisHisHisHisHis-Metal ion, wherein His is histidine, Phe is phenylalanine, p is an integer between 1 and 3, and wherein the Metal ion is as selected herein, e.g., Cu,
NH₂-DOPA-(Phe)₁-(His)₆-OH, wherein Phe is phenylalanine, His is histidine, wherein the amine and hydroxyl groups are the amino acid N and C termini, respectively,
NH₂-DOPA-(Phe)₂-(His)₆-OH, wherein Phe is phenylalanine, His is histidine, wherein the amine and hydroxyl groups are the amino acid N and C termini, respectively,
NH₂-DOPA-(Phe)₁-(His)₃-OH, wherein Phe is phenylalanine, His is histidine, wherein the amine and hydroxyl groups are the amino acid N and C termini, respectively,
NH₂-DOPA-(Phe)₁-(His)₄-OH, wherein Phe is phenylalanine, His is histidine, wherein the amine and hydroxyl groups are the amino acid N and C termini, respectively,
NH₂-DOPA-(Phe)₁-(His)₅-OH, wherein Phe is phenylalanine, His is histidine, wherein the amine and hydroxyl groups are the amino acid N and C termini, respectively,
NH₂-DOPA-(Phe)₂-(His)₃-OH, wherein Phe is phenylalanine, His is histidine, wherein the amine and hydroxyl groups are the amino acid N and C termini, respectively,
NH₂-DOPA-(Phe)₂-(His)₄-OH, wherein Phe is phenylalanine, His is histidine, wherein the amine and hydroxyl groups are the amino acid N and C termini, respectively,
NH₂-DOPA-(Phe)₂-(His)₅-OH, wherein Phe is phenylalanine, His is histidine, wherein the amine and hydroxyl groups are the amino acid N and C termini, respectively,
NH₂-DOPA-(Phe)₁-(His)₆-OH with a metal ion, wherein Phe is phenylalanine, His is histidine, wherein the amine and hydroxyl groups are the amino acid N and C termini, respectively, and wherein the Metal ion is as disclosed herein, e.g., Cu,
NH₂-DOPA-(Phe)₂-(His)₆-OH with a metal ion, wherein Phe is phenylalanine, His is histidine, wherein the amine and hydroxyl groups are the amino acid N and C termini, respectively, and wherein the Metal ion is as disclosed herein, e.g., Cu,
NH₂-DOPA-(Phe)₁-(His)₃-OH with a metal ion, wherein Phe is phenylalanine, His is histidine, wherein the amine and hydroxyl groups are the amino acid N and C termini, respectively, and wherein the Metal ion is as disclosed herein, e.g., Cu,
NH₂-DOPA-(Phe)₁-(His)₄-OH with a metal ion, wherein Phe is phenylalanine, His is histidine, wherein the amine and hydroxyl groups are the amino acid N and C termini, respectively, and wherein the Metal ion is as disclosed herein, e.g., Cu,
NH₂-DOPA-(Phe)₁-(His)₅-OH with a metal ion, wherein Phe is phenylalanine, His is histidine, wherein the amine and hydroxyl groups are the amino acid N and C termini, respectively, and wherein the Metal ion is as disclosed herein, e.g., Cu,
NH₂-DOPA-(Phe)₂-(His)₃-OH with a metal ion, wherein Phe is phenylalanine, His is histidine, wherein the amine and hydroxyl groups are the amino acid N and C termini, respectively, and wherein the Metal ion is as disclosed herein, e.g., Cu,
NH₂-DOPA-(Phe)₂-(His)₄-OH with a metal ion, wherein Phe is phenylalanine, His is histidine, wherein the amine and hydroxyl groups are the amino acid N and C termini, respectively, and wherein the Metal ion is as disclosed herein, e.g., Cu,
NH₂-DOPA-(Phe)₂-(His)₅-OH with a metal ion, wherein Phe is phenylalanine, His is histidine, wherein the amine and hydroxyl groups are the amino acid N and C termini, respectively, and wherein the Metal ion is as disclosed herein, e.g., Cu.

Each of the above compounds constitutes an independent embodiment of compounds of the invention.

Each of the above compounds is further targeted for use in forming an antimicrobial film or coat on a surface region of an object, as discussed herein.

Each of the above compounds, forming an independent embodiment of the invention, is an antimicrobial agent, namely a material having antibacterial, antiviral and/or antifungal properties.

In some embodiments, in each of the above specified materials, independently, the metal ion may be selected from Cu metal ions, Ni metal ions, Au metal ions, Zn metal ions, Ag metal ions, Mg metal ions, Ti metal ions, Co metal ions, Zr metal ions, Sn metal ions, Pb metal ions, Mo metal ions and combinations thereof, and each linker moiety L, independently, may be an organic moiety selected from a substituted or unsubstituted carbon chain, an amino acid and a peptide of 1 to 50 amino acids, or 1 to 20 amino acids, or 2 to 20, or 2 to 10 amino acids or 2, 3, 4, 5, 6, 7, 8, 9, 10 amino acids.

Materials or compounds of the invention exhibit antimicrobial properties, including antiviral and/or antifungal properties and/or antibacterial properties. These may be measured in solution or in a film form, when a material of the invention is formed into film or when the material is self-assembled as a film on a substrate. The “antimicrobial properties”, including antibacterial, antifungal and antiviral properties, encompass antifouling properties, namely minimizing, diminishing, arresting or preventing adhesion of bacteria, fungi and/or viruses to a surface. More specifically, the properties include, inter alia, (i) prevention of accumulation of organisms or organism's secretion, (ii) prevention or arrest of adsorption of secretion products of cells of multicellular organisms or of microorganisms, (iii) prevention of bacterial, and viral adhesion, (iv) prevention of attachment of larger organisms or cells shed from bodies of multicellular organisms, (v) eliminating or decreasing proliferation of microorganisms, (vi) prevention of biofilm generation, formation or growth, (vii) reducing or preventing quorum sensing, (viii) preventing adhesion of molecules and scale formation; (ix) eradicating or killing microorganisms present on the surface or coming into contact with the surface; and others. Putting it differently, compounds of the invention are configured to inhibit settling, attachment, accumulation and dispersion of microorganisms and/or microorganism's secretion and/or organic and/or bio-organic material (e.g., proteins and/or (poly)saccharides and/or (poly)lipids) and/or scale; and/or eliminate or decrease or prevent proliferation of microorganisms on their surface; and/or killing the microorganisms or destroying viral capsid or the fungal/bacterial cell wall.

Thus, materials of the invention may be used for (i) prevention of accumulation of organisms or organism's secretion, (ii) prevention or arrest of adsorption of secretion products of cells of multicellular organisms or of microorganisms, (iii) prevention of bacterial, and viral adhesion, (iv) prevention of attachment of larger organisms or cells shed from bodies of multicellular organisms, (v) eliminating or decreasing proliferation of microorganisms, (vi) prevention of biofilm generation, formation or growth, (vii) reducing or preventing quorum sensing, (viii) preventing adhesion of molecules and scale formation; or for (ix) eradicating or killing microorganisms present on the surface or coming into contact with the surface.

As used herein, the antimicrobial effect attributed to materials of the invention encompasses antiviral, antibacterial, antifungal as well as anti-yeast properties.

As the effect of the metal ion present on the microorganism may not be specific, the metal ion can induce the antimicrobial effect on a wide range of bacteria (Gram positive and negative), viruses and fungi and may be active simultaneously against all three microorganisms or pathogens. For example, materials of the invention may be effective against bacteria such as Staphylococcus aureus, Staphylococcus epidermidis, Escherichia coli, Clostridium difficle, Bacillus subtilis, Pseudomonas aeruginosa, Legionella pneumophila and Lysteria monocytogenes. Similarly, compounds of the invention may be effective against viruses such as bacteriophages, human immunodeficiency virus type 1 (HIV-1), dengue virus, influenza, herpes simplex virus, coxsackievirus, yellow fever virus, respiratory syncytial virus, cytomegalovirus, Canine corona. Examples of fungi include candida, Fusarium, Aspergillus, Myxomycetes, Saccharomyces cerevisiae, molds, Phoma destructiva, Curvularia lunata, Alternaria alternata, Fusarium oxysporum.

As noted herein, materials of the invention may induce antimicrobial properties in solution, in solid state or on a surface. Materials of the invention are particularly suitable for forming stable adherent films or coats on an object surface. Surface coating may be achievable by treating the object surface with a solution of a material or by immersing the object in a solution comprising the material. Thus, films and coatings as well as objects having a surface region coated with such films and coatings are also within the scope of the invention, as further detailed herein.

Without wishing to be bound by theory, a film of a material is formed spontaneously on the object surface due to the adhesive properties of the surface-adhering functionality, e.g., DOPA. The resulting coat or film has the following properties:
it self-assembles into ordered films;
it tightly adheres to the surface;
it is capable of releasing anti-microbial metal ions, thereby inducing antimicrobial properties to the surface;
it may be used as a metal-ion reservoir, wherein the metal ions are not released in bulk but rather provide continuous protection over long period of time;
it is a colorless, transparent and does not have an effect on the substantial optical, chemical or mechanical properties of the surface onto which it is formed;
it is a thin film;
it may be regenerated;
it is biodegradable;
it is environmentally safe; and
it is non-toxic.

Films or coatings may be formed on any object or article of manufacture, being a substantially 2D object, such as a material sheet, or a 3D object. The surface may be of any material, including glass, paper, polymeric (such as polycarbonates, PVC, plastics, silicon etc), metallic (copper, nickel, stainless steel, titanium, etc), and others. The film or coating may be any such coat that covers a region or a complete surface of a substrate. The coat need not be a uniform layer on the entire surface of the object or article of manufacture. As the metal ions are release to the environment in the vicinity of the article or the surface of the article, having the compound of the invention is some regions or in patches and not as a uniform coat may suffice.

Non-limiting examples of objects or articles include countertop, tables, desks, transported dividers or shields cutting surfaces, kitchen appliances, medical appliances and surfaces, surgical tools an apparatuses, masks and visors, gloves, door handles, elevator buttons, keyboard, touchscreens and others.

Objects and article of manufacture having at least one surface region coated with a material of the invention are also within the scope of the present invention.

Sterile objects and articles of manufacture having at least one surface region coated with a material of the invention are further within the scope of the present invention.

The present invention also concerns a process for forming a film of at least one material of the invention on a surface region of an article of manufacture or an object, the process comprising contacting the surface region of the article of manufacture or the object with a solution comprising the material under conditions enabling adhesion of the material to the surface region.

In some embodiments, the article of manufacture or object is pretreated to remove surface impurities. In some embodiments, pretreatment is achieved by oxygen/Plasma cleaning optionally followed by cleaning with agents such as SDS, TDW ethanol, acetone, methanol, and isopropanol.

In some embodiments, adhesion of the material to a surface region is enabled by immersing the object or adding to the surface region to be coated a solution comprising the material. In some embodiments, the immersing or adding is carried out at room temperature at a slightly basic solution, with an increased ionic strength (154 mM NaCl).

In some embodiments, the material is provided when associated to a metal ion.

In some embodiments, the material is provided metal free. In such embodiments, the process may further comprise a step of immersing or treating the surface region coated with the material with a solution of metal ions.

In some embodiments, the solution of metal ions further contains an amount of a reducing agent such as NaBH₄ to reduce some of the metal ions into the corresponding metal, thereby achieving an additional and significant antiviral and antibacterial activities.

Also provided is a kit comprising at least one material of the invention, in a metal-containing form or in a metal-free form, in solution or as a powder, and instructions of use.

The kit may contain the material in an undiluted or solid form and may additionally comprise a solution or a medium for dissolving the material in. The kit may also contain a receptacle for mixing the material and the medium.

The kit may contain a metal-containing material or a metal-free material. Where a metal-free material is provided, e.g., in solution or in a solid form, the kit may further contain a receptacle or vial containing the metal ions in a solid salt or complex form or in solution, and instructions to mix the material and the metal ions at given concentrations.

The invention further provides a kit comprising a material of the invention when associated with a metal ion, as defined, and instructions of use. In some embodiments, the kit further comprises a solution or a medium for forming a solution comprising the material.

The invention further provides a kit comprising one or more containers comprising a material of the invention when free of a metal ion, one or more other containers comprising a metal salt or complex of at least one metal ion(s) and means for mixing the metal ion free material with the metal ion(s) for forming the metal charged materials, and instructions of use.

A further kit is provided that comprises one or more containers comprising a material of the invention when free of a metal ion, one or more other containers comprising a metal salt or complex of at least one metal ion(s) and means for forming a film or a coat of the metal ion free compound on a surface region and further means for contacting the film or coat with the metal ion(s), for forming a film or a coat of the metal charged materials, and instructions of use.

In some embodiments of kits of the invention, the various means may be containers, solvents, solutions, liquid media, brush, spray, a mixing tool or others, as may be required to achieve a desired use.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-C depict formation of an exemplary material according to some embodiments of the invention: (FIG. 1A) The molecular structure of the material, i.e., peptide. (FIG. 1B) A scheme representing the three coating steps. (FIG. 1C) Real-time QCM-D measurements of the peptide and a control peptide without DOPA. The 5th overtones are presented. The arrows indicate the addition of the peptides, washing with tris and the addition of a CuCl₂ solution.

FIGS. 2A-B provide characterization of the P-, PC- and PCN-coated surfaces: (FIG. 2A) SEM images of the peptide network-like nanostructures on P- and PC-coated surfaces. The same structures appear on a PCN-coated surface as well as the appearance, on the same surface, of CuNPs embedded on the peptide nanostructures. (FIG. 2B) EDS analysis of P-, PC-, and PCN-coated surfaces.

FIGS. 3A-C provide composition and structural analysis of the P-, PC-, and PCN-coated surfaces. (FIG. 3A) Atomic concentrations (%) of carbon, nitrogen, titanium and copper on P-, PC-, and PCN-coated surfaces. (FIG. 3B) Area (%) below the Cu(II) related peaks and Cu(O) or Cu(I) related peaks in the Cu 2p spectra for PC and PCN-coated surfaces. (FIG. 3C) FTIR spectra of the peptide, a mixture of the peptide and CuCl₂ and a mixture of the peptide, CuCl₂, and NaBH₄.

FIGS. 4A-D provide transparency of the PCN-coated SiO₂ surface. (FIG. 4A) A photo of a PCN-coated surface indicating the surface's transparency. (FIGS. 4B-C) Photos depicting the contact angles of a clean SiO₂ surface (FIG. 4B) and a PCN-coated SiO₂ surface (FIG. 4C). (FIG. 4D) UV-Vis-NIR transmittance spectra of a clean SiO₂ surface and a PCN-coated surface.

FIGS. 5A-D provide antiviral and antibacterial activity of the peptide-coated surfaces. (FIG. 5A) Virucidal activity of a PCN-coated SiO₂ surface against a clean SiO₂ surface (control). (FIG. 5B) Virucidal activity of P-, PC-, and PN-coated SiO₂ surfaces against a clean SiO₂ surface. (FIG. 5C) Bactericidal activity of a PCN-coated SiO₂ surface against a clean SiO₂ surface. (FIG. 5D) A photo of an agar plate from the antibacterial assay with bacteria from a clean SiO₂ surface (top) and with bacteria from a PCN-coated surface (bottom). Bd=below detection.

FIGS. 6A-C provide a proposed antiviral and antibacterial mechanism of the coating: (FIG. 6A) A calibration curve of Cu(I) ions preformed using a BCA assay. The known Cu(I) concentration data points are depicted in black, their linear trendline in red, and the data point corresponding to the Cu(I) concentration released from the PCN-coated surface is depicted in green. (FIG. 6B) A calibration curve of H₂O₂ was performed using an ABTS assay. The known H₂O₂ concentration data points are depicted in black, their linear trendline in grey, and the data point corresponding to the H₂O₂ concentration released from the new, old, and renewed PCN-coated surfaces are depicted. (FIG. 6C) The suggested mechanism of the antibacterial and antiviral activity of the PCN-coated surface.

### DETAILED DESCRIPTION OF EMBODIMENTS

### Experimental Section

Materials: Fmoc-DOPA(acetonide)-OH, Fmoc-Phe-OH, and Fmoc-His(Trt)-OH were purchased from GL Biochem (Shenghai, China). Triisopropylsilane (TIPS) was purchased from TCI Europe N.V. (Zwijndrecht, Belgium). 1-[Bis(dimethylamino)methylene]-1H-1,2,3-triazolo[4,5-b]pyridinium 3-oxide hexafluorophosphate (HATU) was purchased from Matrix Innovation, Inc. (Saint-Hubert, QC, Canada). Dimethylformamide (DMF), dichloromethane (DCM), piperidine, diethyl ether, N,N-Diisopropylethylamine (DIPEA), acetonitrile, and tri-fluoroacetic acid (TFA) were purchased from Bio-Lab (Jerusalem, Israel). Sodium phosphate monobasic monohydrate was purchased from Mallinckrodt Chemicals (Chesterfield, UK). Difco Nutrient Agar and Difco LB broth, Lennox were purchased from BD Biosciences (San Jose, CA, USA). HCL fuming (37%), tryptic soy broth (TSB), lecithin, and tween 80 were purchased from Merck (Darmstadt, Germany). Lysogeny broth (LB) was purchased from Becton Dickinson (BD) (Franklin Lakes, NJ, USA). Agarose was purchased from Lifegene (Mevo Horon, Israel). Sodium dodecyl sulfate (SDS) was purchased from J.T. Baker Inc. (Phillipsburg, NJ, USA). Pierce BCA protein assay kit was purchased from Thermo Fisher Scientific (Waltham, MA, USA). NaCl was purchased from Fisher Scientific (Hampton, NH, USA). D₂O was purchased from Cambridge Isotope Laboratories, Inc (Tewksbury, MA, USA). NaBH4, CuCl₂, and CuCl were purchased from Acros Organics (Fair Lawn, NJ, USA). 2-Chlorotrityl chloride resin (1.0-1.6 mmol/g, 100-200 mesh) was purchased from Chem-Impex International, Inc (Wood Dale, IL, USA). E. coli (Migula) Castellani and Chalmers strain B (ATCC 11303) and FDA strain Seattle 1946 (ATCC 25922) and E. coli bacteriophage T4 (ATCC 113030-B4) were obtained from American Type Culture Collection (Manassas, VA, USA).

Substrates: Ti substrates: Si wafers with a diameter of 2 inches were coated with 50 nm Ti (as measured by a quartz crystal microbalance) using electron beam evaporation (TFDS-141E, VST) at a rate of 2 Å/sec. Si substrates: Si wafers with a diameter of 10 cm were used. Mica surfaces: mica discs with a 9.9 mm diameter (Ted Pella Inc., Redding, CA, USA) were used. Glass substrates: glass microscope slides (76 mm×26 mm×1 mm, Paul Marienfeld GmbH & CO. KG, Lauda-Königshofen, Germany) were diced into 1×1 cm² pieces before use (7100 2 in. Pro-Vectus, ADT).

Peptide Synthesis: The peptides were synthesized manually by Fmoc solid phase peptide synthesis (SPPS). 2-Chlorotrityl chloride resin was used (0.25 mmol scale). The amino acids were used with a 5-fold excess and they were activated using a DIPEA/HATU mixture (4 equiv. and 3.9 equiv., respectively) for 4 min. The coupling procedure was carried for 1 h and a Kaiser test was performed after each coupling. The Fmoc protecting group was removed by stirring with a solution of 20% piperidine in DMF for 20 min. The washing procedure included washing twice with DMF, methanol, DCM, and DMF again. Before the cleavage of the peptide, the resin was washed three times with DMF and DCM. The cleavage reaction was performed using a mixture of TFA/TIPS/TDW (10 mL, 95:2.5:2.5) for 3 h. The cleaved solution was evaporated via nitrogen bubbling, precipitated with diethyl ether, and centrifuged. The crude peptide product was dissolved in an ACN/TDW mixture (5 mL, 1:1) and lyophilized.

High-Performance Liquid Chromatography (HPLC) Analysis: Analytical reverse-phase HPLC analysis was performed using a Waters Alliance HPLC with UV detection (at 220 and 254 nm) and an XSelect C18 column (3.5 μm 130 Å, 4.6 mm×150 mm). The peptides were eluted using a linear gradient of ACN (with 0.1% TFA) in TDW (with 0.1% TFA), at a flow rate of 1 mL/min, 30° C.

Liquid Chromatography Mass Spectrometry (LC/MS) Analysis: LC(UV)MS/MS analysis was performed using Agilent 6520 Q-TOF analyzer (Agilent Technologies, Santa Clara, CA, USA).

Surface Modification: Mica disks were cleaved before each use. 1×1 cm² Ti or Si surfaces were sonicated for 15 min in ethanol, washed with TDW, and dried with nitrogen. 1×1 cm² SiO₂ surfaces were cleaned with Oxygen/Plasma (Atto, Diener Electronic) for 10 min, immersed in SDS (2%) for 30 min, washed with TDW and dried under N₂. Immediately before coating with the peptide, the surfaces were treated by 02 plasma for 1 min (Atto, Diener Electronic, Ebhausen, Germany). The surfaces were immersed in the peptide solution (1.5 mg/mL, 1.1 mM, in a filtered Tris buffer at pH=8.5, 10 mM, with an ionic strength of 154 mM with NaCl) for 4 h, at room temperature. The coated surfaces were washed three times with 1 mL of the same Tris buffer solution and dried with nitrogen. After that, the surfaces were immersed in the CuCl₂ solution (0.8 mg/mL, 4.6 mM, in the same Tris buffer solution) for 2 h, at room temperature. The washing step was repeated. Lastly, the surfaces were immersed in an ice-cold NaBH₄ solution (0.3 mg/mL, 9.1 mM in the same Tris buffer) for 1 h, at room temperature, and the washing step was repeated.

Quartz Crystal Microbalance with Dissipation (QCM-D) Analysis: The peptides' adsorption and the metal-binding capabilities were studied using QCM-D (Q-sense, Biolin Scientific). The measurements were performed in a flow module E1 system with SiO₂ sensors with a fundamental resonant frequency of 5 MHz (Qsense). The sensors were cleaned according to the supplier instructions. The experiments were performed under flow-through conditions using a digital peristaltic pump (IsmaTec Peristaltic Pump, IDEX). The peptide (1.5 mg/mL), control peptide (1.5 mg/mL), and CuCl₂ (0.8 mg/mL) were dissolved in a filtered Tris buffer solution (pH=8.5, 10 mM). The solutions were injected circularly into the sensor crystal chamber at a rate of 0.1 mL/min. The peptide solution flowed for -4 h, the control peptide for −80 min, and the CuCl₂ solution for -2 h. Between and after the coatings, the sensor was washed with the same Tris buffer solution. The adsorbed mass was calculated according to the Sauerbrey equation using the 5th overtones.

High Resolution Scanning Electron Microscopy (HR-SEM) and Energy-Dispersive X-ray Spectroscopy (EDS) Analyses: Si and mica surfaces were cleaned and coated in the same methods mentioned above. The mica surface was coated with Ir using Quorum Q150V S Plus Sputter Coater. sputter coater. HR-SEM images were taken using Sirion XL30 SFEG (Thermo Fisher, former FEI), operating at 5 kV, and equipped with XMAX SDD EDS detector (Oxford Instruments, Abingdon, UK) on an Inca Energy 450 platform. Extra-high-resolution SEM (XHR-SEM) images were acquired using Magellan 400L (Thermo Fisher, former FEI), operating at 2 kV.

Atomic Force Microscopy (AFM): Mica surfaces were cleaned as mentioned above and one surface was coated using the three-step procedure. AFM images were taken in NanoWizard 3 instrument (JPK, Berlin, Germany) at AC mode with a Si tip that has a spring constant of 6 N/m (Aspire, Team Nanotec GmbH, Villingen-Schwenningen, Germany).

X-ray Photoelectron Spectroscopy (XPS) Analysis: XPS measurements were taken using Kratos AXIS Supra spectrometer (Kratos Analytical Ltd., Manchester, UK). The spectra were acquired using the Al-Kα monochromatic radiation X-ray source (1486.6 eV). The sample takeoff angle was 900 (normal to the analyzer) and the vacuum pressure in the analyzing chamber was maintained at 2×10⁻⁹ Torr. High-resolution XPS spectra were collected for C 1s, N 1s, Ti 2p, O 1s, and Cu 2p with pass energy 20 eV and 0.1 eV step size. The binding energies were calibrated using C is peak energy as 285.0 eV. The data were analyzed using ESCApe processing program (Kratos Analytical Ltd.) and Casa XPS (Casa Software Ltd.). The thickness of a coated Si surface was calculated based on the formula:

$$d = {λ}_{0} sin θ ln ( \frac{{N}_{s} {λ}_{s} {I}_{0}}{{N}_{0} {λ}_{0} {I}_{s}} + 1 )$$

where d is the thickness in nm, I0 and Is are the intensities of the peaks from the layer and the substrate, respectively, the substrate is the 2p signal from Si and the layer is the sum of the intensities of C 1s, O 1s, and N 1s peaks, θ is the takeoff angle (here sin θ=1), and NO and Ns are the volume densities. The inelastic mean free paths parameters for the layer XO and the substrate kλ were assumed as 3.3 nm and 3.09 nm, respectively (calculated using QUASES-IMFP-TPP2M software).

Attenuated Total Reflectance Fourier-Transform Infrared (ATR-FTIR) Spectroscopy Analysis: The spectra of a bare Ti surface, as well as p-, PC-, and PCN-coated Ti surfaces was measured using a Nicolet 6700 FTIR spectrometer (Thermo Fisher Scientific, Waltham, MA, USA) with Ge-ATR arrangement (VariGATR, Harrick Scientific, Pleasantville, NY, USA). The experimental setup for all spectra included 3000 scans, an applied force of 350 N, an incident angle of 65°, and 4 cm⁻¹ resolution.

Fourier-Transform Infrared (FTIR) Spectroscopy Analysis: A peptide solution (1.5 mg/mL), a 1:1 mixture of the peptide solution and a CuCl₂ solution (0.8 mg/mL), and a 1:1:1 mixture of the peptide, CuCl₂, and NaBH₄ (0.3 mg/mL) solutions were deposited on CaF₂ plates and dried under vacuum. The peptide and mixtures on the plates were resuspended in D₂O and dried under vacuum twice in order to remove the H₂O hydration layer from the plate. The infrared spectra were recorded using a Nicolet 6700 FTIR spectrometer with a deuterated triglycine sulfate (DTGS) detector (Thermo Fisher Scientific, Waltham, MA, USA). The experimental setup included 2000 scans and 4 cm⁻¹ resolution. The absorbance peaks were determined using the OMNIC analysis software (Nicolet).

Contact Angle Measurements: Contact angle measurements were taken using a Theta Lite optical tensiometer (Attention, Finland). Each result is composed of an average of 3 repeats with 20 measurements on each surface.

Transmittance Measurements: The absorption spectra of a clean SiO₂ surface and a SiO₂ surface coated with the peptide, CuCl₂, and treated with NaBH₄ were measured using a UV/Vis spectrophotometer (Shimadzu, UV-1650PC, Kyoto, Japan). The transmittance (% T) of each wavelength was calculated from the absorbance (A) according to the relation: % T=10^{(2-A)}.

Plaque Assay: The assay was based on ISO 21702 standard with several changes. Three clean SiO₂ surfaces and three SiO₂ surfaces coated with the peptide, CuCl₂, and treated with NaBH4 were used in each of the three experiments. For the control experiments, three surfaces coated with just the peptide, the peptide and CuCl₂, and the peptide with NaBH₄ treatment were used. A single colony of E. coli 11303 was transferred to 10 mL nutrient broth (2% LB broth) and incubated overnight at 37° C., 120 rpm. From a heated nutrient agar solution (0.5% NaCl), 2 mL was transferred to each of the wells in 6-wells plates and left to solidify. E. coli bacteriophage T4 stock solution was diluted to a concentration of 106 plaque-forming units (PFU)/mL in LB phage (0.8% LB broth, 0.5% NaCl). From the bacteriophage solution, 16 μL were dropped on each sample and a 0.8×0.8 cm² parafilm was placed on top to evenly spread the bacteriophages on the surface. The samples were incubated at room temperature for 24 h. The phages were removed from the surfaces after 24 h by washing 3 times with SCDLP broth (2 mL). The samples were shaken in the SCDLP broth (3% TSB, 0.1% lecithin, and 0.7% tween 80) for 15 min at 150 rpm. A 10-fold dilution was prepared from each sample to a dilution of 10⁻¹. Warm agarose (0.6%, 1 mL), the E. coli starter (25 μL), and the appropriate washed bacteriophages solutions (20 μL) were added into test tubes. Each tube was gently shaken, and its contents poured over the previously prepared agar plates. The plates were left at room temperature for a few minutes until the agarose solidified and placed at 37° C. in an incubator overnight. The plates were then removed from the incubator, the plaques from each well were counted, and the PFUs were determined according to the dilution.

Antibacterial Assay: The assay was based on JIS Z 2801 standard with several changes. Three clean SiO₂ surfaces and three SiO₂ surfaces coated with the peptide, CuCl₂, and treated with NaBH₄ were used in each of the three experiments. A single colony of E. coli 25922 was transferred to TSB solution (20 mL) and incubated overnight at 37° C., 120 rpm. The solution was centrifuged at 4000 rpm for 10 min, the supernatant was removed, and the precipitate resuspended in PBS buffer (10 mM, pH=7.0, 154 mM NaCl, 1% TSB, 10 mL). This stage was repeated for three more times. The optical density (OD) of the solution was measured at 600 nm using a UV/Vis spectrophotometer (Shimadzu, UV-1650PC, Kyoto, Japan) and the bacteria concentration was diluted accordingly to 106 colony forming units (CFU)/mL. A 20 μL drop from the bacteria solution was deposited in the middle of each surface and covered with a 0.8×0.8 cm² parafilm layer. The surfaces were incubated for 24 h in a humid atmosphere at 37° C. Afterwards, the surfaces were placed in test tubes with PBS buffer (1 mL). Each test tube was separately sonicated for 1 min and vortexed for 15 see, then 10⁻¹ to 10⁻³ dilutions were prepared using PBS buffer, and 20 μL stripes were pipetted on agar plates (0.5% NaCl). The plates were incubated overnight at 37° C., the colonies were counted and the CFUs were determined according to the dilution.

Statistics: The data from the plaque and antibacterial assays represent three independent experiments. In each plaque assay experiment, three uncoated and three coated surfaces were used. Each plaque assay was performed in duplicates and each antibacterial assay was performed in triplicates. The results for the control plaque assay, as well as the assays meant to display the effect of NaCl, were produced from one experiment. All the graphs were plotted using Origin software (OriginLab Corporation) and the results were compared using Student's two sample t-test assuming unequal variances. Statistically significant results were marked with one asterisk for P<0.01 (*) and two asterisks for P<0.001 (**).

Bicinchoninic acid (BCA) Assay: A CuCl solution (0.1 mg/mL) was prepared by first dissolving the CuCl in HCl (37%) and diluting the solution in TDW. This solution was then diluted to a total of 8 different CuCl concentrations. A drop of TDW (100 μL) was placed on a SiO₂ surface that was coated with the peptide, CuCl₂, and treated with NaBH₄. The surface was left for 24 h in a humid environment. The Pierce BCA protein assay kit reagents A and B were combined at a 50:1 ratio and 200 μL were added from this mixture to 25 μL from the 8 CuCl dilutions and to 25 μL from the water deposited on the coated surface after 24 h. The mixtures were gently mixed, incubated at 37° C. for 30 min, cooled down to room temperature and their absorbance was measured at 562 nm using a UV/Vis spectrophotometer (Shimadzu, UV-1650PC, Kyoto, Japan). A calibration curve was made using the CuCl dilutions and the Cu(I) concentration released from the coated surface to the water was calculated.

### Peptide Design

A variety of peptide compounds according to the invention have been prepared and studied. An exemplary compound in the form of a trifunctional peptide NH₂-DOPA-(Phe)₂-(His)₆-OH is described herein.

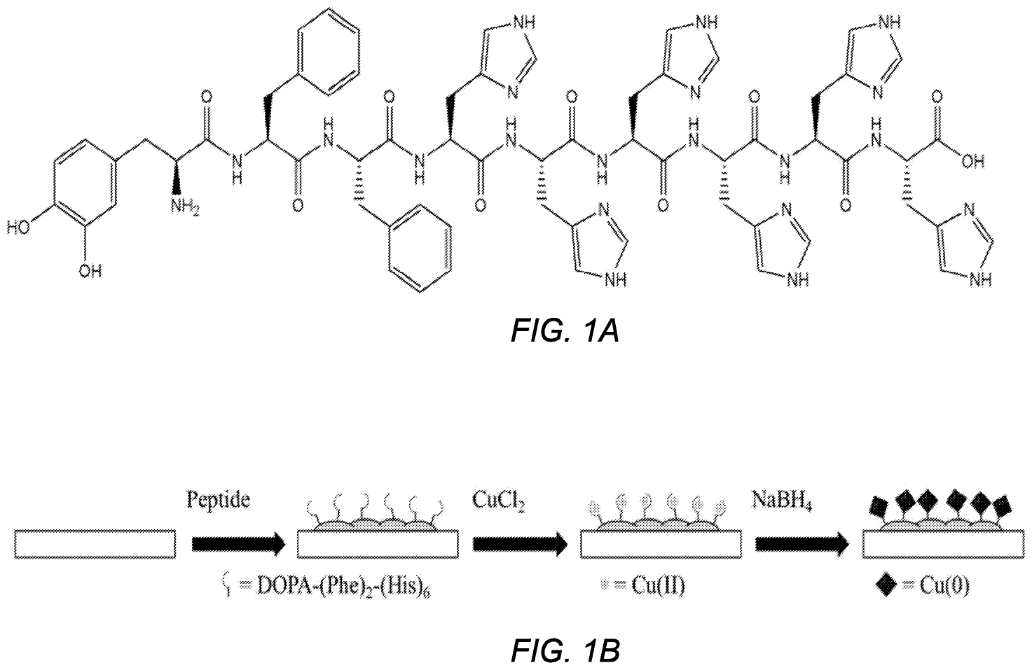

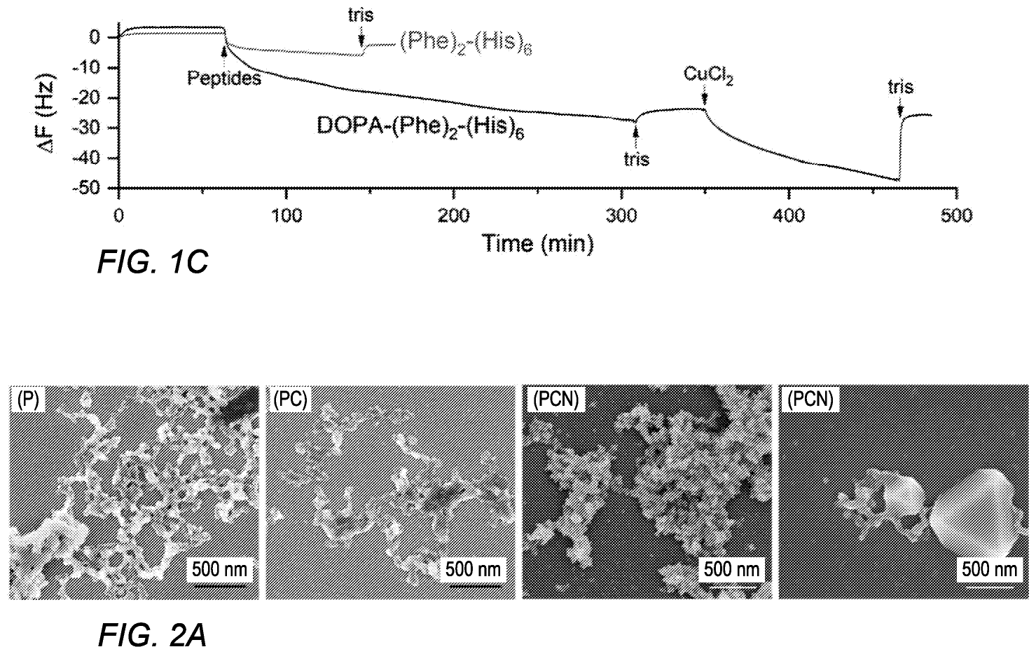

The peptide design incorporates three elements: (i) the amino acid DOPA, which provides the peptide with adsorption capabilities to most substrates; (ii) a diphenylalanine segment, which facilitates the peptide's self-assembly process through π stacking, and which may be optional for some of the peptide compounds, and (iii) a hexahistidine unit, for generating a coordinative association various metal ions, e.g., Ni²⁺ and Cu²⁺ (FIG. 1A). Since Cu was proved to possess antiviral and antibacterial capabilities through various pathways, it was chosen for this research. Employment of a single peptide sequence with the three functions mentioned is highly beneficial in designing an antiviral and antibacterial coating.

### Surface Modification

Surface modification was performed using a three-step procedure in which the surfaces were immersed in a peptide solution (P), a Cu solution (PC), and an NaBH₄ solution (PCN) to reduce the copper (FIG. 1B). These steps were meant to allow the formation of a peptide layer on the surface, form bonds between the histidine residues of the peptide and Cu ions, and finally reduce these ions to cover the peptide layer with Cu crystals. The three steps were performed in a Tris buffer solution with an ionic strength of 154 mM with NaCl. After every immersion, the surface was rinsed three times with 1 mL of the buffer solution, to ensure the removal of weakly-attached residues and to preliminarily demonstrate the stability of the coating.

The oxidation of DOPA is considered to assist its adhesion by allowing multiple interactions with the surface. To promote this process, the procedure was performed at alkaline conditions using Tris buffer. Additionally, these conditions were favored because at lower pH values the imidazole groups on the histidine residues are protonated and can no longer bind metal ions.

The interactions of the salt ions with water could abate the interactions between water and the polar amino acids, i.e., histidine and DOPA. This could encourage the coacervation of the peptide via π stacking interactions between the phenylalanine residues, thus forming a peptide layer more easily in a solution with a higher ionic strength. The effect of the salt on the coacervation of the peptide can be seen in the solutions of the peptide, Cu(II), and their mixture, with and without NaCl. While no turbidity can be seen in the solutions without NaCl, the coacervation is clearly visible in the peptide-Cu mixture with NaCl. Additionally, NaCl is used in His-tag purification as it can reduce weak ionic interactions and prevent nonspecific binding, which could be formed in this case between the peptide and the surface or other components in the solution.

The adhesion of the peptide and its ability to bind Cu ions were investigated by quartz crystal microbalance with dissipation (QCM-D) analysis. The peptide flowed for ˜4 h and caused a substantial change in frequency, which indicated the adsorption of the peptide to the SiO₂ sensor (FIG. 1C). After washing the sensor with the buffer solution, only a small change in frequency was observed, which points to the stability of the peptide coating. The changes in dissipation were ˜1×10⁻⁶, which suggests the formation of a rigid film and supports the use of the Sauerbrey equation to resolve the adsorbed mass on the sensor. The loaded mass/area for the peptide was ˜96 ng/cm². Afterwards, a Cu(II) solution was introduced and caused a large change in frequency, which decreased after washing with the buffer. However, a smaller change remained, corresponding to a loaded mass/area value of ˜9 ng/cm². This value is expected in view of the lower mass of the Cu ions bound to the peptide and it corresponds to a ratio of ˜2 Cu ions per each peptide molecule, as is expected from previous studies.

To demonstrate the importance of DOPA for the peptide's adhesion, we synthesized the peptide NH₂-(Phe)₂-(His)₆-OH (without DOPA) and investigated its adhesion to the surface using QCM-D. This peptide caused a small frequency change, which was further reduced by washing with the buffer. The loaded mass/area for this peptide was ˜12 ng/cm², an eighth of the value for the DOPA-containing peptide, which indicates the significant effect of DOPA to the adhesion.

### Surface Characterization

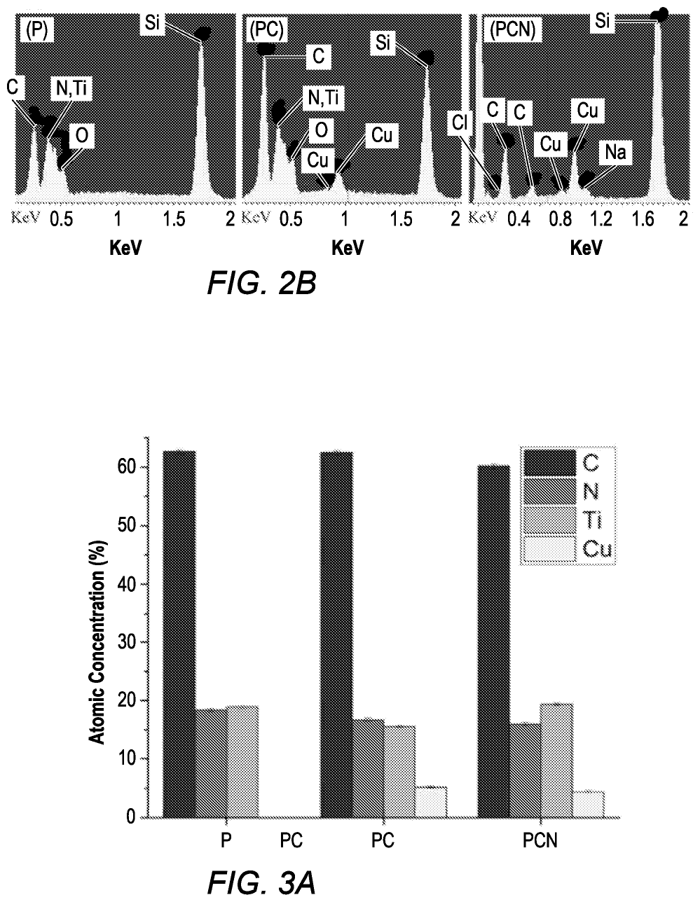

Surfaces coated with the peptide (P), peptide and Cu (PC), and those coated with the peptide and Cu and treated with NaBH₄ (PCN) were analyzed using scanning electron microscopy (SEM). The SEM images show a network-like nanostructure of the peptide (FIG. 2A). Similar structures were previously received from a hexahistidine peptide's assembly with Zn ions. The structure does not differ greatly with the addition of Cu ions but appears rougher after the reduction treatment with NaBH₄. Additionally, structures that appear to be Cu nanoparticles (CuNPs) embedded in the peptide network-like nanostructures were visible on the PCN-coated surface. Energy-dispersive X-ray Spectroscopy (EDS) analysis showed the presence of the peptide on the surface and of Cu bound to the peptide, which were not diminished after treatment with NaBH₄ (FIG. 2B).

To view the differences between a clean surface to areas that are not covered with the peptide nanostructures on a PCN-coated surface, freshly cleaved and PCN-coated mica surfaces were imaged using atomic force microscopy (AFM). The topography of the bare mica was unobservable due to the low signal-to-noise ratio, while that of the PCN-coated surface was distinct. The difference can be seen also from the different roughness parameters (Rq) measured for the bare mica surface (0.3 nm) and for the PCN-coated surface (1.3 nm).

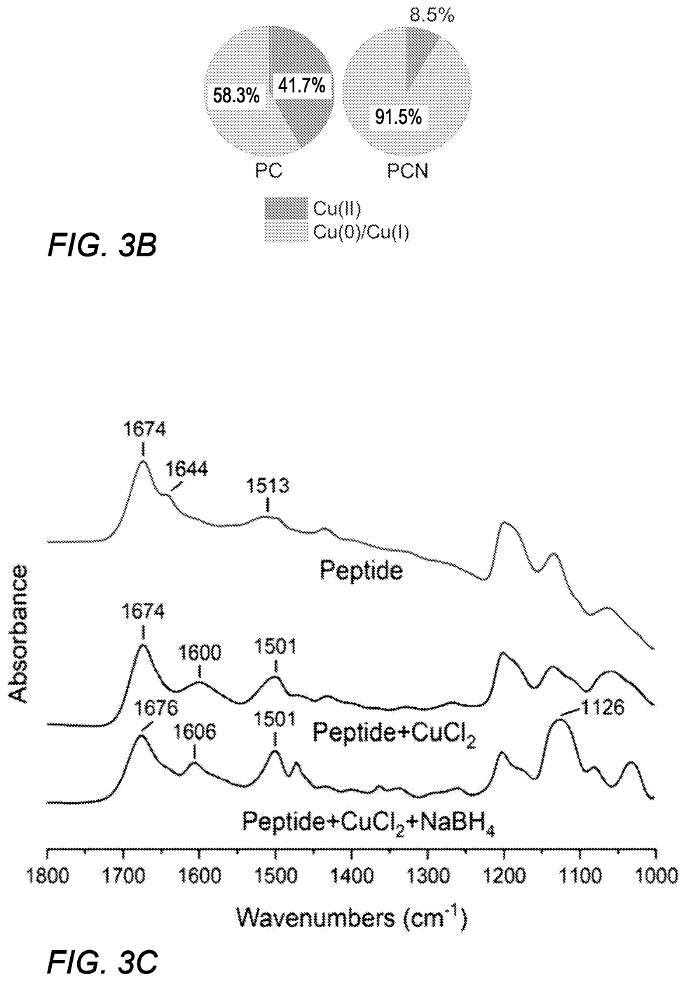

X-ray Photoelectron Spectroscopy (XPS) was used to further examine P-, PC-, and PCN-coated surfaces. The results supported the presence of the peptide and of the Cu on the surfaces (FIG. 3A). The reduction step with NaBH₄ did not appear to remove the peptide layer. Additionally, the resulting Cu 2p spectra indicated that the reduction step was successful as it increased the area below the Cu(0) and Cu(I) related peaks from 58% to 91% at the expense of the area below the Cu(II) related peaks (FIG. 3b). This also suggested that there is some level of Cu(IJ) reduction done by the peptide itself, before introducing the reducing agent. In addition, the XPS measurements allow to calculate the average thickness of the PCN coating, which was established at 6.5 nm.

To decipher the secondary structure of the peptide on the surface, attenuated total reflectance Fourier-transform infrared (ATR-FTIR) spectroscopy measurements of a clean Ti surface and P-, PC-, and PCN-coated Ti surfaces were performed. As a result of the low signal of the peptide, no secondary structure could be inferred from the spectra. This could be explained if the thickness of the coating was on the range of a few nanometers. However, the formation of a coating can be derived from the disappearance of the bare Ti peak at 876 cm⁻¹ on the coated surfaces. Because of the lower signal in ATR-FTIR, we performed FTIR analyses that have a higher signal-to-noise ratio. We measured CaF₂ plates coated with the peptide, a mixture of the peptide and CuCl₂, and a mixture of the peptide, CuCl₂, and NaBH₄. The strong peaks at 1674 cm⁻¹ for P- and PC-coated plates and at 1676 cm⁻¹ for PCN-coated plate indicate that the peptide formed p-turns, which were unaffected by the addition of CuCl₂ and NaBH₄ (FIG. 3C). A lower peak at 1644 in the P-coated plate could associated with a disordered structure of the peptide or of random coil structures. These structures appear to diminish when Cu(II) ions are added, suggesting that the ions can strengthen the interactions present in the peptide structures causing the 3-turn structures to be predominant. The broad peak at 1600 cm-1 and the narrower one at 1606 cm⁻¹ for PC- and PCN-coated surfaces, respectively, could be attributed to the degree of crystallinity of the structures on the plate. This could be explained due to the formation of Cu crystals on the PC- and PCN-coated plates and is supported by the XPS results, which show a majority of reduced Cu atoms also on the PC-coated surface (FIG. 3B). The peak at 1513 cm⁻¹ in the spectrum of the P-coated plate could be indicative of imidazole ring stretching (FIG. 3C). The shift and narrowing of this peak at the spectra of the PC- and PCN-coated plates could point to interactions between the imidazole ring of the histidine residues and the Cu. Additionally, the peak at 1126 cm⁻¹ in the spectrum of the PCN-coated plate could be associated with the stretching vibration of CN in the imidazole. These data support the formation of peptide structures and the existence of the peptide's interactions with Cu ions or with metallic Cu through the histidine residues.

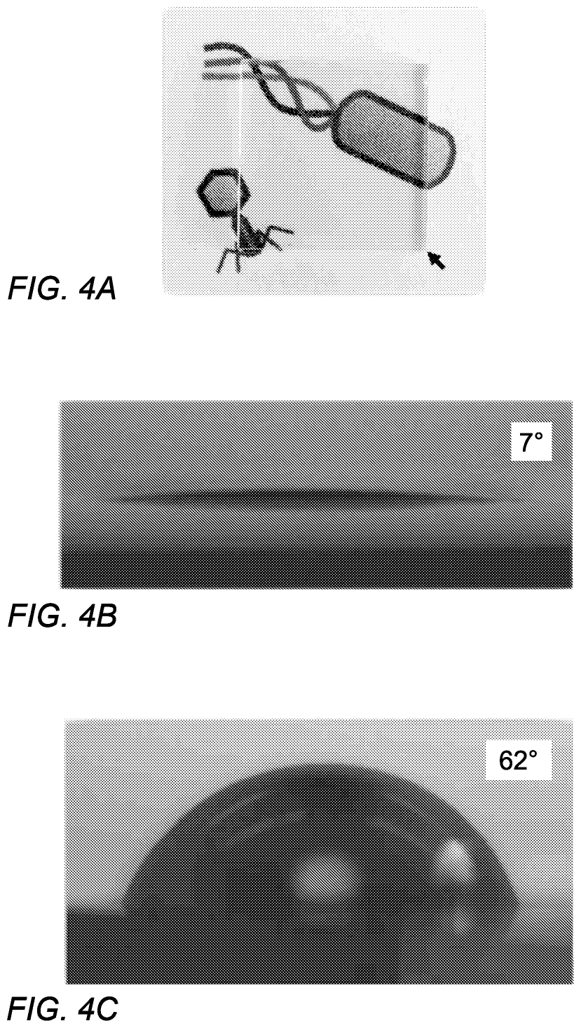

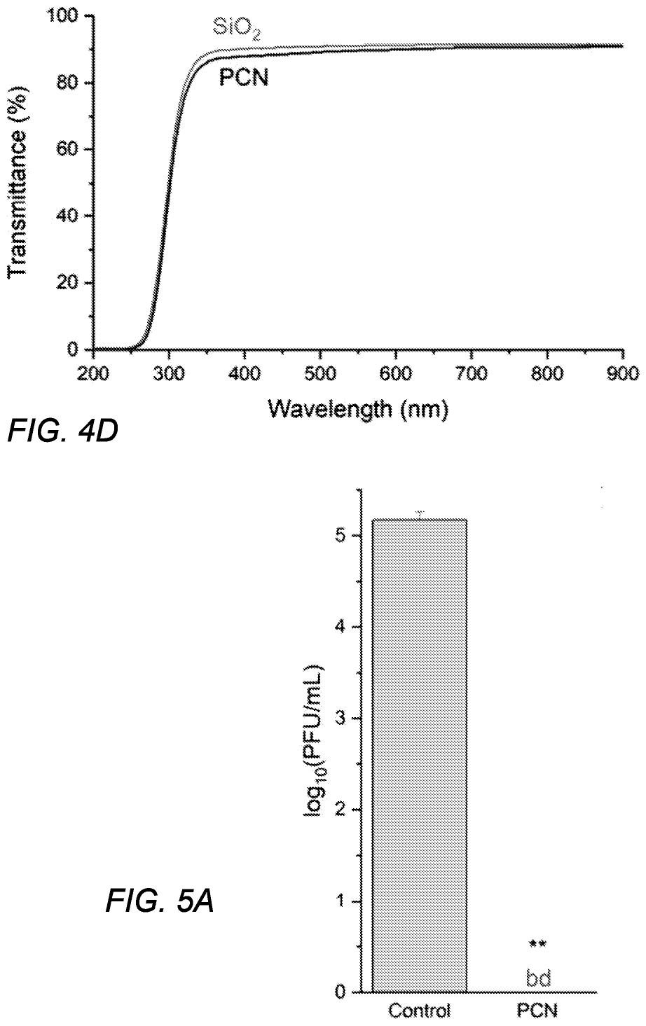

The coating procedure resulted in an optically transparent coating, as expected from the coating's thickness (FIG. 4A). The difference between a clean surface and the coated surface, while not visible to the naked eye, can be revealed in contact angle measurements. A clean glass surface presented a contact angle of 7° and the coated surface showed an increase of the contact angle to 620 (FIG. 4B,C). Transmittance spectra of a clean glass surface and a PCN-coated surface were measured and confirmed the transparency of the coating in the UV-Vis-NIR light range (FIG. 4D).

### Antiviral Activity

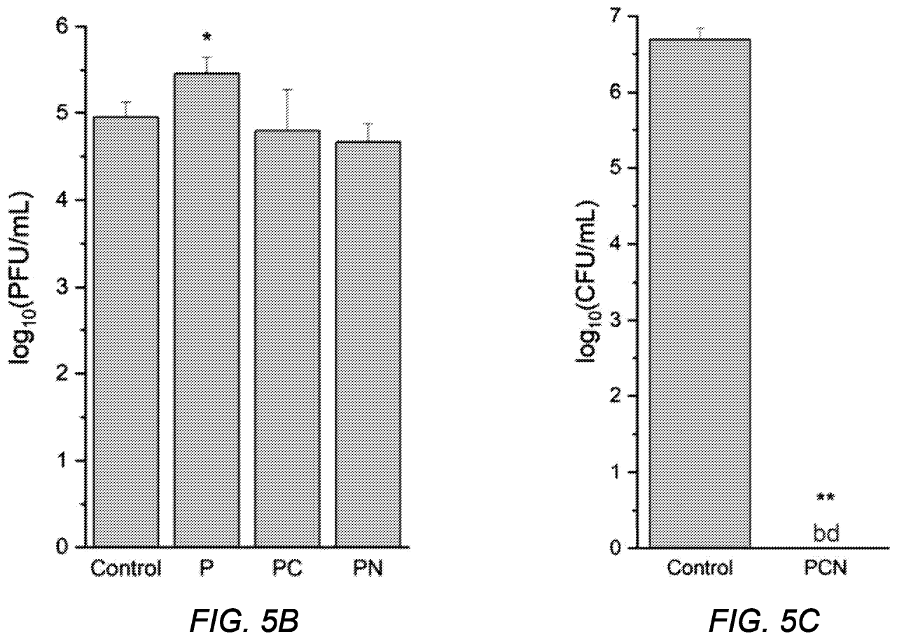

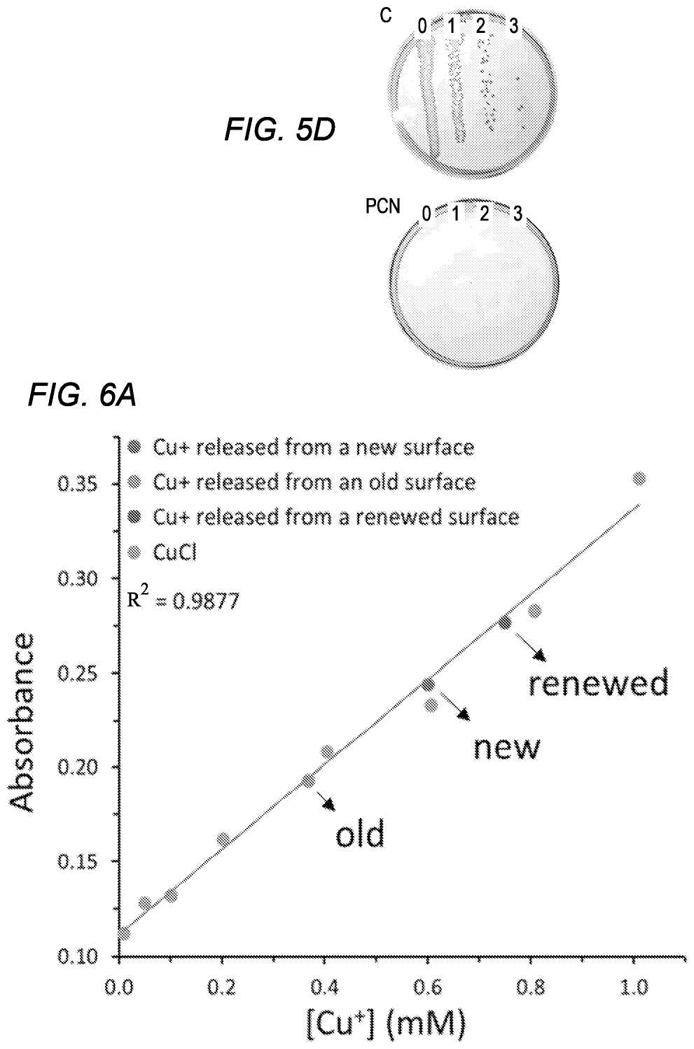

To assess the antiviral activity of the PCN-coated surface we performed a plaque assays using T4 bacteriophages (ATCC 113030-B4) and Escherichia coli (ATCC 11303) as their host. The amount of bacteriophage plaques after incubation on a coated surface, when compared to those that were cast on uncoated surfaces, can provide insight to the coating's antiviral and virucidal capabilities. As presented in FIG. 5A, the coating reduced the number of phages by more than 5 orders of magnitude (100%) when compared to uncoated SiO₂ surfaces.

To identify the necessary factors in the coating for the antiviral activity, P- and PC-coated SiO₂ surfaces, as well as surfaces coated with the peptide and treated with NaBH₄ (PN) were also examined with bacteriophage plaque assays. From these assays, presented in FIG. 5B, it appears that the peptide coating by itself could promote the survivability of the phages on the surface. This may be explained due to the emergence of niches in the peptide's network structure, in which the phages can shelter from the exposed SiO₂ surface and the potential evaporation of the phage solution. On the other hand, PC- and PN-coated surfaces did not display an explicit reduction of the bacteriophage numbers. The results for these surfaces were not consistent, resulting in relatively large standard deviation values, which strengthens their inferiority to the PCN-coated surface.

The importance of the salt to the antiviral activity of the coating can be inferred from the plaque assays of PCN-coated surfaces with and without NaCl in the buffer solutions. Without NaCl the number of phages was reduced by 39% and with NaCl the number was reduced by 80%. These assays were performed on Ti surfaces, with 3 h incubation time of the surfaces with the phages instead of 24 h.

### Antibacterial Activity

The PCN-coated surface was tested for bactericidal activity on E. coli (ATCC 25922). The number of bacterial colonies on the coated surface was reduced by more than 6 orders of magnitude (100%) when compared to a clean SiO₂ surface (FIG. 5C). These results, when combined with the virucidal activity, reveal the effectiveness of the coating's antiviral and antibacterial capabilities (FIG. 5D).

### Suggested Antiviral and Antibacterial Mechanism

Different Cu materials display a pleiotropic effect as they involve several antiviral and antimicrobial pathways, negating the development of antibiotic resistance. The exact antiviral and antimicrobial mechanism of Cu remains uncertain, but several processes have been proposed. Cu ions were shown to bind bacterial cells, causing membrane depolarization that leads to leaks and ruptures. Another process associated with Cu ions, which is also apparent in CuNPs, involves the production of ROS that can damage the genetic material and the lipid membranes. Specifically, Cu(II) ions have a lesser effect on viruses while the more potent Cu(I) ions were shown to possess significant antiviral abilities. The antiviral and antimicrobial abilities of metallic Cu via contact killing were proposed to involve four processes: (i) Cu dissolution from the surface causes cell damage, (ii) the cellular membrane loses its integrity and its contents, (iii) the Cu ions induce ROS formation, and (iv) the genetic material undergoes degradation.

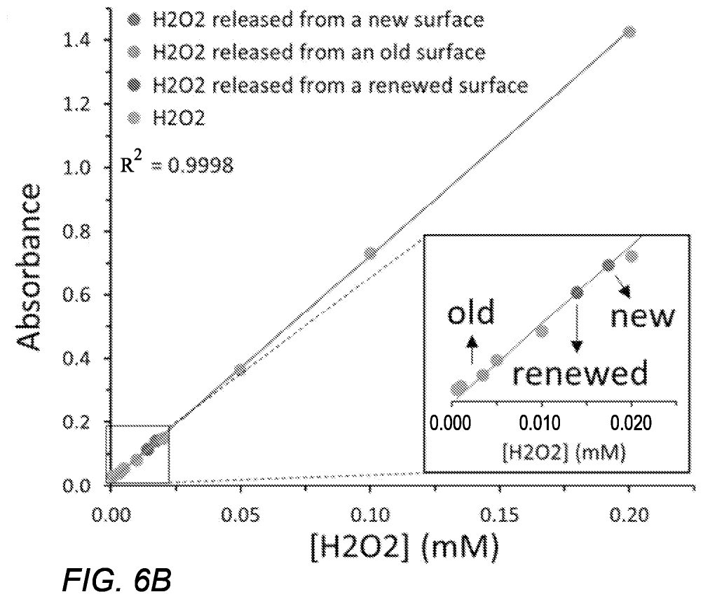

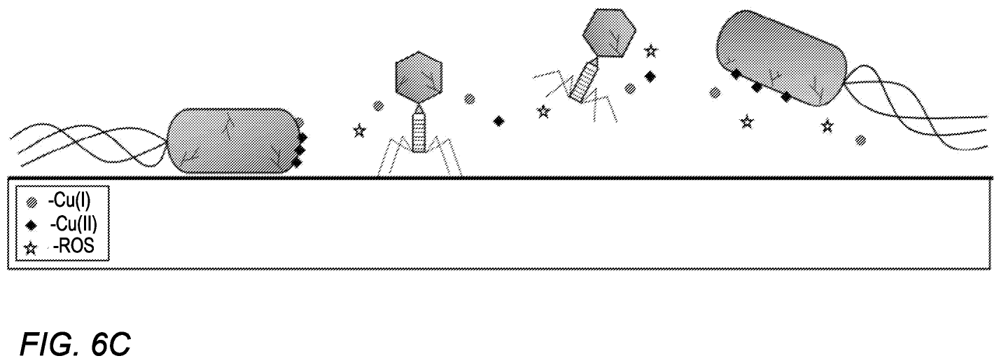

To clarify the origin of the coating's antiviral and antibacterial activity, we performed an assay that will reveal the presence of Cu(I) ions released from the surface. The assay was carried out with bicinchoninic acid (BCA) that forms a complex with Cu(I) ions, which retains a prominent linear absorption at 562 nm. A calibration curve was made using CuCl solutions with known concentrations and the concentration of Cu(I) released from a PCN-coated surface was shown to be 0.2 mM (FIG. 6A,B). Since Cu(I) has well studied antibacterial and antiviral properties, this result could provide insight to the mechanism of those properties in the PCN coating (FIG. 6C). The inclusion of the potent antiviral and antibacterial abilities of Cu(I) ions to the possible dissolution of ROS generating Cu(II) ions and to the properties of metallic Cu and CuNPs could result in a strong antiviral and antibacterial coating that kills both viruses and bacteria.

1-49. (canceled)

50. An antimicrobial material comprising at least one surface adhering functionality, at least one organic metal-ion associating functionality, and optionally at least one structurally and/or biologically effective functionality, excluding F-based functionalities selected from fluorinated alkyls, fluorinated aryls, and F atoms.

51. The material according to claim 50 being of any one of the general formulae:
A-M, or
A-L-M, or
A-M-T, or
T-A-M, or
A-T-M, or
T-A-L-M, or
A-L-T-M, or
A-T-L-M, or
A-L-M-T;
wherein A is the at least one surface adhering functionality, M is the at least organic metal-ion associating functionality, L is at least one linker moiety, T is the at least one structurally and/or biologically effective functionality and “-” is at least one bond.

52. The material according to claim 50, wherein the at least one surface adhering functionality is a catechol-based functionality.

53. The material according to claim 50, wherein the at least one surface adhering functionality is 3,4-dihydroxy-L-phenylalanin (DOPA).

54. The material according to claim 50, wherein the at least one organic metal-ion associating functionality is at least one organic functionality comprising an atom or a group of atoms capable of reversibly associating to a metal ion.

55. The material according to claim 50, wherein the at least one metal-ion associating functionality consists or comprises histidine.

56. The material according to claim 55, wherein the metal-ion associating functionality is a peptide consisting or comprising -HisHisHisHisHisHis.

57. A material comprising at least one surface adhering functionality, at least one organic metal-ion associating functionality comprising between 3 and 10 histidine amino acid units or comprising a peptide functionality consisting between 3 and 10 histidine units, and optionally at least one structurally and/or biologically effective functionality.

58. A material having antimicrobial properties, the material comprising at least one surface adhering functionality, at least one organic metal-ion associating functionality consisting between 3 and 10 histidine amino acid units, and optionally at least one structurally and/or biologically effective functionality.

59. The material according to claim 1, the material comprising
at least one surface adhering functionality,
at least one organic metal-ion associating functionality comprising between 3 and 10 histidine amino acid units or comprising a peptide functionality consisting between 3 and 10 histidine units, wherein the metal-ion associating functionality coordinatively associates a metal ion selected from Cu metal ions, Ni metal ions, Au metal ions, Zn metal ions, Ag metal ions, Mg metal ions, Ti metal ions, Co metal ions, Zr metal ions, Sn metal ions, Pb metal ions, Mo metal ions and combinations thereof, and
optionally at least one structurally and/or biologically effective functionality.

60. The material according to claim 50, wherein the at least one structurally and/or biologically effective functionality is an amino acid having a pendent nitrogen group.

61. The material according to claim 60, wherein the amino acid is lysine.

62. The material according to claim 50, the material being any one compound of the following:
DOPA-(His)y, wherein His is histidine and y is an integer between 3 and 10,
(DOPA)x-(His)y, wherein x is an integer between 1 and 3, His is histidine and y is an integer between 3 and 10,
(DOPA)x-(AAA)m-(His)y, wherein x is an integer between 1 and 3, AAA is an aromatic amino acid, m is an integer between 1 and 3, His is histidine and y is an integer between 3 and 10,
DOPA-(Phe)p-(His)y, wherein Phe is phenylalanine, p is an integer between 1 and 3, His is histidine and y is an integer between 3 and 10,
DOPA-HisHisHis, wherein His is histidine,
DOPA-HisHisHisHis, wherein His is histidine,
DOPA-HisHisHisHisHis, wherein His is histidine,
DOPA-HisHisHisHisHisHis, wherein His is histidine,
DOPA-HisHisHis-Metal ion, wherein His is histidine,
DOPA-HisHisHisHis-Metal ion, wherein His is histidine,
DOPA-HisHisHisHisHis-Metal ion, wherein His is histidine,
DOPA-HisHisHisHisHisHis-Metal ion, wherein His is histidine,
DOPA-L-HisHisHis, wherein His is histidine, and wherein L is a linker moiety,
DOPA-L-HisHisHisHis, wherein His is histidine, and wherein L is a linker moiety,
DOPA-L-HisHisHisHisHis, wherein His is histidine, and wherein L is a linker moiety,
DOPA-L-HisHisHisHisHisHis, wherein His is histidine, and wherein L is a linker moiety,
DOPA-L-HisHisHis-Metal ion, wherein His is histidine, L is a linker moiety,
DOPA-L-HisHisHisHis-Metal ion, wherein His is histidine, L is a linker moiety,
DOPA-L-HisHisHisHisHis-Metal ion, wherein His is histidine, L is a linker moiety,
DOPA-L-HisHisHisHisHisHis-Metal ion, wherein His is histidine, L is a linker moiety,
DOPA-(Phe)p-HisHisHis, wherein His is histidine, Phe is phenylalanine, and wherein p is an integer between 1 and 3,
DOPA-(Phe)p-HisHisHisHis, wherein His is histidine, Phe is phenylalanine, and wherein p is an integer between 1 and 3,
DOPA-(Phe)p-HisHisHisHisHis, wherein His is histidine, Phe is phenylalanine, and wherein p is an integer between 1 and 3,
DOPA-(Phe)p-HisHisHisHisHisHis, wherein His is histidine, Phe is phenylalanine, and wherein p is an integer between 1 and 3,
DOPA-(Phe)p-HisHisHis-Metal ion, wherein His is histidine, Phe is phenylalanine, p is an integer between 1 and 3,
DOPA-(Phe)p-HisHisHisHis-Metal ion, wherein His is histidine, Phe is phenylalanine, p is an integer between 1 and 3,
DOPA-(Phe)p-HisHisHisHisHis-Metal ion, wherein His is histidine, Phe is phenylalanine, p is an integer between 1 and 3,
DOPA-(Phe)p-HisHisHisHisHisHis-Metal ion, wherein His is histidine, Phe is phenylalanine, p is an integer between 1 and 3,
(Phe)p-DOPA-HisHisHis, wherein His is histidine, Phe is phenylalanine, and wherein p is an integer between 1 and 3,
(Phe)p-DOPA-HisHisHisHis, wherein His is histidine, Phe is phenylalanine, and wherein p is an integer between 1 and 3,
(Phe)p-DOPA-HisHisHisHisHis, wherein His is histidine, Phe is phenylalanine, and wherein p is an integer between 1 and 3,
(Phe)p-DOPA-HisHisHisHisHisHis, wherein His is histidine, Phe is phenylalanine, and wherein p is an integer between 1 and 3,
(Phe)p-DOPA-HisHisHis-Metal ion, wherein His is histidine, Phe is phenylalanine, p is an integer between 1 and 3,
(Phe)p-DOPA-HisHisHisHis-Metal ion, wherein His is histidine, Phe is phenylalanine, p is an integer between 1 and 3,
(Phe)p-DOPA-HisHisHisHisHis-Metal ion, wherein His is histidine, Phe is phenylalanine, p is an integer between 1 and 3,
(Phe)p-DOPA-HisHisHisHisHisHis-Metal ion, wherein His is histidine, Phe is phenylalanine, p is an integer between 1 and 3,
NH₂-DOPA-(Phe)₁-(His)₆-OH, wherein Phe is phenylalanine, His is histidine, wherein the amine and hydroxyl groups are the amino acid N and C termini, respectively,
NH₂-DOPA-(Phe)₂-(His)₆-OH, wherein Phe is phenylalanine, His is histidine, wherein the amine and hydroxyl groups are the amino acid N and C termini, respectively,
NH₂-DOPA-(Phe)₁-(His)₃-OH, wherein Phe is phenylalanine, His is histidine, wherein the amine and hydroxyl groups are the amino acid N and C termini, respectively,
NH₂-DOPA-(Phe)₁-(His)₄-OH, wherein Phe is phenylalanine, His is histidine, wherein the amine and hydroxyl groups are the amino acid N and C termini, respectively,
NH₂-DOPA-(Phe)₁-(His)₅-OH, wherein Phe is phenylalanine, His is histidine, wherein the amine and hydroxyl groups are the amino acid N and C termini, respectively,
NH₂-DOPA-(Phe)₂-(His)₃-OH, wherein Phe is phenylalanine, His is histidine, wherein the amine and hydroxyl groups are the amino acid N and C termini, respectively,
NH₂-DOPA-(Phe)₂-(His)₄-OH, wherein Phe is phenylalanine, His is histidine, wherein the amine and hydroxyl groups are the amino acid N and C termini, respectively,
NH₂-DOPA-(Phe)₂-(His)₅-OH, wherein Phe is phenylalanine, His is histidine, wherein the amine and hydroxyl groups are the amino acid N and C termini, respectively,
NH₂-DOPA-(Phe)₁-(His)₆-OH with a metal ion, wherein Phe is phenylalanine, His is histidine, wherein the amine and hydroxyl groups are the amino acid N and C termini, respectively,
NH₂-DOPA-(Phe)₂-(His)₆-OH with a metal ion, wherein Phe is phenylalanine, His is histidine, wherein the amine and hydroxyl groups are the amino acid N and C termini, respectively,
NH₂-DOPA-(Phe)₁-(His)₃-OH with a metal ion, wherein Phe is phenylalanine, His is histidine, wherein the amine and hydroxyl groups are the amino acid N and C termini, respectively,
NH₂-DOPA-(Phe)₁-(His)₄-OH with a metal ion, wherein Phe is phenylalanine, His is histidine, wherein the amine and hydroxyl groups are the amino acid N and C termini, respectively,
NH₂-DOPA-(Phe)₁-(His)₅-OH with a metal ion, wherein Phe is phenylalanine, His is histidine, wherein the amine and hydroxyl groups are the amino acid N and C termini, respectively,
NH₂-DOPA-(Phe)₂-(His)₃-OH with a metal ion, wherein Phe is phenylalanine, His is histidine, wherein the amine and hydroxyl groups are the amino acid N and C termini, respectively,
NH₂-DOPA-(Phe)₂-(His)₄-OH with a metal ion, wherein Phe is phenylalanine, His is histidine, wherein the amine and hydroxyl groups are the amino acid N and C termini, respectively,
NH₂-DOPA-(Phe)₂-(His)₅-OH with a metal ion, wherein Phe is phenylalanine, His is histidine, wherein the amine and hydroxyl groups are the amino acid N and C termini, respectively,
wherein in each of the materials, independently, the metal ion is selected from Cu metal ions, Ni metal ions, Au metal ions, Zn metal ions, Ag metal ions, Mg metal ions, Ti metal ions, Co metal ions, Zr metal ions, Sn metal ions, Pb metal ions, Mo metal ions and combinations thereof, and wherein each linker moiety L is an organic moiety selected from a substituted or unsubstituted carbon chain, an amino acid and a peptide of 1 to 50 amino acids, or 1 to 20 amino acids, or 2 to 20, or 2 to 10 amino acids or 2, 3, 4, 5, 6, 7, 8, 9, 10 amino acids.

63. The material according to claim 50 for forming a film or a coat on a surface.

64. The material according to claim 50, for use in a method of (i) prevention of accumulation of organisms or organism's secretion, (ii) prevention or arrest of adsorption of secretion products of cells of multicellular organisms or of microorganisms, (iii) prevention of bacterial, and viral adhesion, (iv) prevention of attachment of larger organisms or cells shed from bodies of multicellular organisms, (v) eliminating or decreasing proliferation of microorganisms, (vi) prevention of biofilm generation, formation or growth, (vii) reducing or preventing quorum sensing, (viii) preventing adhesion of molecules and scale formation; or (ix) eradicating or killing microorganisms present on the surface or coming into contact with the surface.

65. A film of a material according to claim 50.

66. An object or an article of manufacture having a surface region coated with a film according to claim 65.

67. A kit comprising at least one material according to claim 50 in a metal-containing form or in a metal-free form, in solution or as a powder, and instructions of use.
